(19) United States
(12) Patent Application Publication
(10) Pub. No.: US 2025/0072838 A1
(43) Pub. Date: Mar. 6, 2025

(54) VASCULAR ACCESS DEVICES, SYSTEMS, AND METHODS

(71) Applicant: Veris Health, Inc., New York, NY (US)

(72) Inventors: James D. Mitchell, New York, NY (US); Andrew A. Thoreson, New York, NY (US); Lishan Aklog, New York, NY (US); Brian J. deGuzman, New York, NY (US); Richard Yazbeck, New York, NY (US); Stephen J. O'Neill, New York, NY (US)

(21) Appl. No.: 18/691,965

(22) PCT Filed: Sep. 20, 2022
(86) PCT No.: PCT/US22/76731

§ 371 Date: Mar. 14, 2024

Related U.S. Application Data
(60) Provisional application No. 63/261,454, filed on Sep. 21, 2021.
(60) Provisional application No. 63/262,989, filed on Oct. 25, 2021.

(51) Int. Cl. A61B 5/00; A61B 5/0205; A61M 25/00; A61M 39/02

(52) U.S. Cl. CPC A61B 5/6865; A61B 5/02055; A61M 25/0026; A61M 39/0247; A61M 2025/0008; A61M 2039/0258; A61M 2039/0267; A61M 2039/0273

(57) ABSTRACT

Vascular access devices and associated systems and methods are disclosed herein. According to some embodiments, the present technology includes an implantable vascular access device comprising a catheter and a housing defining a fluid reservoir and an outlet port configured to mate with a catheter, the outlet port fluidically coupled to the fluid reservoir. The vascular access device can comprise one or more sensing elements carried by the housing and/or the catheter, which are configured to capture physiological data while the device is implanted within a patient The device can also include a data communications unit configured to receive physiological data from the one or more sensing elements and transmit the physiological data to one or more remote computing devices.

### CROSS-REFERENCE TO RELATED APPLICATIONS

This application claims the benefit of the following prior applications:
U.S. Provisional Application No. 63/261,454, filed Sep. 21, 2021, and
U.S. Provisional Application No. 63/262,989, filed Oct. 25, 2021.

The foregoing applications are incorporated by reference herein in their entireties. To the extent the foregoing applications or any other material incorporated herein by reference conflicts with the present disclosure, the present disclosure controls.

### TECHNICAL FIELD

The present technology relates to implantable medical devices and associated systems and methods of use. Particular embodiments of the present technology are directed to vascular access devices, systems, and methods.

### BACKGROUND

Vascular access devices (e.g., vascular access ports) are minimally invasive, surgically implanted devices that provide relatively quick and easy access to a patient's central venous system for the purpose of administering intravenous medications, such as chemotherapeutic agents. Conventional vascular access devices are commonly used for patients requiring frequent, repeated intravenous administration of therapeutic agents or fluid, repeated blood draws, and/or for patients with difficult vascular access.

Vascular access devices such as vascular access ports typically include a reservoir attached to a catheter. The entire unit is placed completely within a patient's body using a minimally invasive surgical procedure. In most cases, the reservoir is placed in a small pocket created in the upper chest wall just inferior to the clavicle, and the catheter is inserted into the internal jugular vein or the subclavian vein with the tip resting in the superior vena cava or the right atrium. However, such vascular access devices can be placed in other parts of the body and/or with the catheter positioned in alternative sites as well. In conventional devices, the reservoir is typically bulky such that the overlying skin protrudes, allowing a clinician to use palpation to localize the device for access when it is to be used for a medication infusion or aspiration of blood for testing. A self-sealing cover (e.g., a thick silicone membrane) is disposed over and seals the reservoir, allowing for repeated access using a non-coring (e.g., Huber type) needle that is inserted through the skin and into the port. This access procedure establishes a system in which there is fluid communication between the needle, the vascular access device, the catheter, and the vascular space, thereby enabling infusion of medication or aspiration of blood via a transcutaneous needle.

Conventional vascular access devices are bulky by design to allow a clinician to localize the device by palpation. To be accurately accessed by a clinician, the vascular access device needs to be either visualized or palpated under the skin. Additionally, conventional vascular access ports have no electronic components and no internal power source. Accordingly, there is a need for improved vascular access devices.

### SUMMARY

The subject technology is illustrated, for example, according to various aspects described below, including with reference to FIGS. 1-37. Various examples of aspects of the subject technology are described as numbered clauses (1, 2, 3, etc.) for convenience. These are provided as examples and do not limit the subject technology.

1. A vascular access device comprising:
a hub comprising a housing defining a reservoir configured to receive a fluid therein;
a catheter comprising a proximal end portion configured to be secured to the hub, a distal end portion configured to be positioned within a cardiovascular system of a patient, and a lumen extending therethrough, the lumen being in fluid communication with the reservoir;
a sensing element carried by the catheter, the sensing element being configured to obtain data characterizing at least one of a physiological parameter of a patient or a performance parameter of the vascular access device; and
a data communications module communicatively coupled to the sensing element and configured to transmit the data obtained by the sensing element to an external computing device.

2. The vascular access device of Clause 1, wherein the data communications module is carried by the housing of the hub.

3. The vascular access device of Clause 1 or Clause 2, wherein the sensing element is configured to communicate with the data communications module via a wireless connection.

4. The vascular access device of Clause 1 or Clause 2, further comprising a conductive element having a first portion electrically coupled to the sensing element and a second portion electrically coupled to the data communications module.

5. The vascular access device of Clause 4, wherein the conductive element and catheter are coextruded.

6. The vascular access device of Clause 4 or Clause 5, wherein the conductive element is at least partially positioned within a sidewall of the catheter.

7. The vascular access device of any one of Clauses 4 to 6, wherein the catheter comprises a channel that is radially offset from the lumen, and wherein the conductive element is positioned within the channel.

8. The vascular access device of any one of Clauses 1 to 7, further comprising a battery in electrical communication with the sensing element.

9 The vascular access device of any one of Clauses 1 to 8, wherein the sensing element is at least partially positioned within a sidewall of the catheter.

10. The vascular access device of any one of Clauses 1 to 9, wherein the sensing element is at least partially exposed to the lumen of the catheter and/or an environment surrounding the catheter.

11. The vascular access device of any one of Clauses 1 to 10, wherein the lumen is a first lumen, the catheter having a second lumen radially offset from the first lumen.

12. The vascular access device of Clause 11, wherein the sensing element is positioned within the second lumen.

13. The vascular access device of Clause 11 or Clause 12, wherein the first lumen is fluidically isolated from the second lumen.

14. The vascular access device of any one of Clauses 1 to 13, wherein the physiological parameter of the patient comprises at least one of a heart rate of the patient, a central venous pressure of the patient, a respiratory rate of the patient, a respiratory sound of the patient, a cardiac sound of the patient, a gastrointestinal sound of the patient, a speech of the patient, a core temperature of the patient, an electrical signal of a heart of the patient, an activity level of the patient, a blood oxygenation of the patient, or a blood glucose of the patient.

15. The vascular access device of any one of Clauses 1 to 14, wherein the performance parameter of the vascular access device comprises at least one of a flow rate within the lumen of the catheter, a pressure in the lumen of the catheter, a temperature of the catheter, an electrical impedance of the sensing element, or a charge level of a battery of the device.

16. A vascular access device comprising:
a hub comprising a housing defining a reservoir configured to receive a fluid therein;
a catheter comprising a proximal end portion secured to the hub, a distal end portion opposite the proximal end portion along a length of the catheter, an intermediate portion therebetween, and a sidewall defining a lumen fluidically coupled to the reservoir; and
a sensing element carried by the intermediate portion of the catheter, the sensing element being configured to obtain data characterizing at least one of a physiological parameter of a patient or a performance parameter of the vascular access device,
wherein the distal end portion of the catheter is separable from the intermediate portion of the catheter such that the length of the catheter is adjustable.

17. The vascular access device of Clause 16, wherein the distal end portion of the catheter is configured to be cut to be separated from the intermediate portion of the catheter.

18. The vascular access device of Clause 16 or Clause 17, wherein the catheter comprises one or more markers configured to indicate a location of at least one of the intermediate portion of the catheter or the distal end portion of the catheter.

19. The vascular access device of Clause 18, wherein the one or more markers comprise at least one of a film, a coating, a surface treatment, a recess, an opening, or a protrusion.

20. The vascular access device of Clause 18 or Clause 19, wherein the one or more markers comprise at least one of a number, a letter, a color, a symbol, a pattern, or a shape.

21. A vascular access device for implanting into a body of a patient, the vascular access device comprising:
a hub comprising a housing defining a reservoir configured to receive a fluid therein; and
a catheter comprising:
   a sidewall defining a lumen configured to be fluidically coupled to the reservoir;
   a proximal end portion configured to be secured to the hub, the proximal end portion having a first length;
   a distal end portion opposite the proximal end portion along a longitudinal axis of the catheter, the distal end portion having a second length; and
   an intermediate portion therebetween, the intermediate portion carrying a sensing element configured to obtain data characterizing at least one of a physiological parameter of a patient or a performance parameter of the vascular access device,
   wherein at least one of the first and second lengths is configured to be modified such that, when the vascular access device is implanted in the patient's body, the sensing element is located at an intended position relative to a specific region of the patient's body.

22. The vascular access device of Clause 21, the intermediate portion further comprising a data communications module communicatively coupled to the sensing element.

23. The vascular access device of Clause 21 or Clause 22, further comprising a data communications module carried by the hub and configured to communicate wirelessly with an external computing device.

24. The vascular access device of Clause 22 or Clause 23, wherein the data communications module is communicatively coupled to the sensing element via a conductive element.

25. The vascular access device of Clause 24, wherein the conductive element has a proximal end portion configured to be electrically coupled to the data communications module and a distal end portion configured to be electrically coupled to the sensing element.

26. The vascular access device of Clause 24 or Clause 25, wherein a first portion of the conductive element is positioned within the sidewall of the catheter and a second portion of the conductive element extends away from the sidewall of the catheter.

27. The vascular access device of Clause 26, wherein the conductive element extends from the first portion within the sidewall to the second portion through an aperture in the sidewall.

28. The vascular access device of Clause 26 or Clause 27, wherein the first portion is the distal end portion of the conductive element, and the second portion is the proximal end portion of the conductive element.

29. The vascular access device of any one of Clauses 25 to 28, wherein the proximal end portion of the conductive element is configured to be electrically coupled to the data communications module via an electrical connector carried by the housing of the hub.

30. A vascular access system comprising:
a hub comprising a housing defining a reservoir configured to receive a fluid therein;
a catheter comprising a proximal end portion configured to be secured to the hub, a distal end portion configured to be positioned within a blood vessel and/or heart of a patient, and a lumen extending therethrough, the lumen being in fluid communication with the reservoir;
a sensing element carried by the catheter and/or the hub, the sensing element being configured to obtain audio data; and
a controller configured to be communicatively coupled to the sensing element and configured to determine at least one of a physiological parameter of the patient, a device parameter, or a treatment parameter based on the audio data.

31. The vascular access system of Clause 30, wherein the physiological parameter comprises at least one of a heart rate, a heart rhythm, a respiratory rate, a pulmonary function testing parameter, a breath sound, a heart sound, an abdominal sound, or a vocal sound.

32. The vascular access system of Clause 30 or Clause 31, further comprising a data communications module communicatively coupled to the sensing element and configured to transmit the data obtained by the sensing element to an external computing device.

33. The vascular access system of any one of Clauses 30 to 32, wherein the controller is carried by the hub.

34. The vascular access system of any one of Clauses 30 to 32, wherein the controller is separate from the hub.

35. The vascular access system of Clause 34, wherein the controller is configured to be extracorporeally positioned when the hub is implanted.

36. The vascular access system of any one of Clauses 30 to 35, wherein the system further comprises an electronics component carried by the hub.

37. The vascular access system of any one of Clauses 30 to 36, wherein the sensing element is a microphone.

38. The vascular access system of any one of Clauses 30 to 37, wherein the sensing element comprises a plurality of microphones.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic representation of a system for monitoring the health of a patient via an implanted medical device in accordance with the present technology.

FIG. 2 shows an example of a vascular access device configured for use with the system of FIG. 1 and in accordance with the present technology.

FIG. 3 shows a vascular access device in accordance with the present technology implanted within a patient's body.

FIG. 4 shows a vascular access device in accordance with the present technology implanted within a patient's body.

FIG. 5 shows a vascular access device in accordance with the present technology implanted within a patient's body.

FIG. 6A is a side cross-sectional view of a portion of a catheter in accordance with the present technology.

FIGS. 6B and 6C are axial cross-sectional views of the catheter of FIG. 6A, taken along lines 6B-6B and 6C-6C, respectively.

FIG. 7A is a side cross-sectional view of a portion of a catheter in accordance with the present technology.

FIGS. 7B and 7C are axial cross-sectional views of the catheter of FIG. 7A, taken along lines 7B-7B and 7C-7C, respectively.

FIGS. 8-13 are axial cross-sectional views of catheters in accordance with the present technology.

FIG. 14A is a side cross-sectional view of a portion of a vascular access device in accordance with the present technology.

FIG. 14B is an axial cross-sectional view of the vascular access device of FIG. 14A, taken along line 14B-14B.

FIG. 15A is a side cross-sectional view of a portion of a vascular access device in accordance with the present technology.

FIG. 15B is an axial cross-sectional view of the vascular access device of FIG. 15A, taken along line 15B-15B.

FIG. 16A is a side view of a catheter in accordance with the present technology.

FIG. 16B is an axial cross-sectional view of the catheter of FIG. 16A, taken along line 16B-16B.

FIGS. 17-24 are axial cross-sectional views of catheters in accordance with the present technology.

FIG. 25 is a side cross-sectional view of a catheter in accordance with the present technology.

FIG. 26 schematically depicts a vascular access device in accordance with the present technology.

FIG. 27 schematically depicts a vascular access device in accordance with the present technology.

FIG. 28 schematically depicts a vascular access device in accordance with the present technology.

FIG. 29 schematically depicts a vascular access device in accordance with the present technology.

FIGS. 30A-30C schematically depict a vascular access device in accordance with the present technology.

FIGS. 31A-31C schematically depict a portion of a catheter of a vascular access device in accordance with the present technology.

FIGS. 32A-32C schematically depict a portion of a catheter of a vascular access device in accordance with the present technology.

FIG. 33 schematically depicts a hub of a vascular access device in accordance with the present technology.

FIGS. 34A and 34B schematically depict a reservoir portion and an electronics portion of a vascular access device in accordance with the present technology.

FIGS. 35A and 35B schematically depict a reservoir portion and an electronics portion of a vascular access device in accordance with the present technology.

FIG. 36 schematically depicts a medical device implanted within a patient in accordance with the present technology.

FIG. 37 schematically depicts a vascular access device in accordance with the present technology.

FIG. 38 is a perspective view of a portion of a catheter in accordance with the present technology.

FIG. 39 is a partially transparent perspective view of a portion of the catheter shown in FIG. 38 with a transverse cross-section of the catheter highlighted.

### DETAILED DESCRIPTION

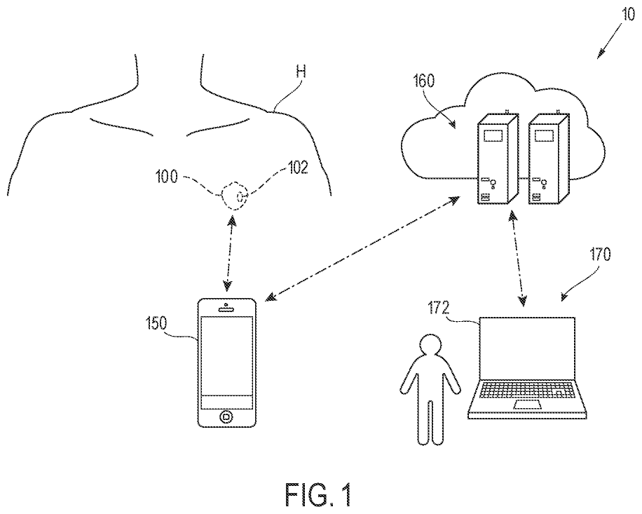

The present technology relates to implantable medical devices such as vascular access devices and associated systems and methods of use. Specific details of several embodiments of the technology are described below with reference to FIGS. 1-37.

The vascular access devices and systems of the present technology may be equipped with electronic components that provide a platform for remote monitoring of the device and/or patient. For example, several of the vascular access devices disclosed herein include a sensing element configured to obtain data characterizing a patient's health, performance of the device, treatment status, and/or other parameters for enhancing patient care. According to some embodiments, a vascular access device can include a sensing element configured to obtain patient physiological data while the vascular access device is implanted within the patient, and determine one or more physiological parameters based on the data. The system may determine certain physiological parameters, for example, that indicate one or more symptoms of a medical condition that requires immediate medical attention or hospitalization. Such physiological parameters can include those related to temperature, patient movement/activity level, heart rate, respiratory rate, blood oxygen saturation, and/or other suitable parameters described herein. Additionally or alternatively, the systems of the present technology can be configured to determine one or more device performance parameters. For example, a vascular access device can include a sensing element configured to obtain data characterizing a flow rate within the device and, based on the data, the system can determine if the device is occluded and/or the extent of the occlusion.

According to various embodiments of the present technology, a vascular access device comprises a hub including a fluid reservoir and a catheter configured to be secured to the hub and fluidically coupled to the reservoir. The vascular access device can include one or more sensing elements carried by the hub and/or one or more sensing elements carried by the catheter. As detailed herein, a sensing element carried by the catheter may be configured to obtain data characterizing certain device parameters and/or physiological parameters and transmit the data to the hub and/or an extracorporeal location (e.g., to an interrogation device, a remote computing device, etc.) for storage and/or further processing. Catheter-level sensing can be particularly beneficial for measuring physiological parameters, as catheters can access more distal and/or central locations within the patient's heart and vasculature and thus provide greater accuracy for certain measurements, such as core temperature, central venous pressure, heart rate, etc. Additionally or alternatively, a sensing element carried by the catheter can be configured to obtain data characterizing catheter performance, as described in greater detail herein.

FIG. 1 is a schematic representation of a system 10 for remote monitoring via a vascular access device 100 (or “device 100”) in accordance with the present technology. The device 100 can be any of the vascular access devices disclosed herein. The device 100 is configured to be implanted within a human patient H, such as at a subcutaneous location along an upper region of the patient's chest. For example, according to several embodiments, the device 100 can be implanted in a subcutaneous pocket created in the patient's upper chest wall, just inferior to the clavicle. As shown in FIG. 1, the device 100 may include a sensing element 102 configured to obtain data characterizing a physiological parameter of the patient, device performance, a status of the treatment administered to the patient, and/or other parameters. In some embodiments, the sensing element 102 can be configured to obtain physiological measurements that are used by the system 10 to determine one or more physiological parameters indicative of the patient's health. For example, the system 10 may detect a medical condition or associated symptom(s) based on the physiological parameter(s) and, optionally, provide an indication of the detected condition to the patient, caregiver, and/or medical care team. Additionally or alternatively, the sensing element 102 can be configured to obtain data used by the system 10 to determine one or more performance parameters indicative of a health and functioning of the device 100. For example, the sensing element 102 can be configured to detect a pressure or a flow rate within the device 100 such that the system 10 is configured to detect occlusion of the device 100 and, optionally, indicate to a patient, caregiver, and/or medical care team that the device 100 needs repair, replacement, and/or removal. In some embodiments, the sensing element 102 is configured to obtain data used by the system 10 to determine one or more treatment parameters, such as time and/or date of infusions, infusion volume, etc. In at least some examples, a diagnostic system and/or method may include delivering a known substance (e.g., saline) one or more times at a known flow rate through the port, and measuring the pressure in the device 100 via one or more sensing elements 102 to diagnose or detect occlusions (e.g., thrombus formation) and/or to confirm patency of the port and/or catheter.

As shown schematically in FIG. 1, the device 100 may be configured to communicate wirelessly with a local computing device 150, which can be, for example, a smart device (e.g., a smartphone, a tablet, or other handheld device having a processor and memory), a special-purpose interrogation device, or other suitable device. Communication between the device 100 and the local computing device 150 can be mediated by, for example, near-field communication (NFC), infrared wireless, Bluetooth, ZigBee, Wi-Fi, inductive coupling, capacitive coupling, or any other suitable wireless communication link. The device 100 may transmit data including, for example, measurements obtained via the sensing element 102 characterizing physiological parameters of the patient, patient medical records, device performance metrics (e.g., battery level, error logs, etc.), or any other such data obtained by or stored by the device 100. In some embodiments, the transmitted data is encrypted or otherwise obfuscated to maintain security during transmission to the local computing device 150. The local computing device 150 may also provide instructions to the vascular access device 100, for example to obtain certain physiological measurements via the sensing element 102, to emit a localization signal, or to perform other functions. In some embodiments, the local computing device 150 may be configured to wirelessly recharge a battery of the device 100, for example via inductive charging.

The system 10 may further include first remote computing device(s) 160 (or server(s)), and the local computing device 150 may in turn be in communication with first remote computing device(s) 160 over a wired or wireless communications link (e.g., the Internet, public and private intranet, a local or extended Wi-Fi network, cell towers, the plain old telephone system (POTS), etc.). The first remote computing device(s) 160 may include one or more own processor(s) and memory. The memory may be a tangible, non-transitory computer-readable medium configured to store instructions executable by the processor(s). The memory may also be configured to function as a remote database, i.e., the memory may be configured to permanently or temporarily store data received from the local computing device 150 (such as one or more physiological measurements or parameters and/or other patient information).

In some embodiments, the first remote computing device(s) 160 can additionally or alternatively include, for example, server computers associated with a hospital, a medical provider, medical records database, insurance company, or other entity charged with securely storing patient data and/or device data. At a remote location 170 (e.g., a hospital, clinic, insurance office, medical records database, operator's home, etc.), an operator may access the data via a second remote computing device 172, which can be, for example a personal computer, smart device (e.g., a smartphone, a tablet, or other handheld device having a processor and memory), or other suitable device. The operator may access the data, for example, via a web-based application. In some embodiments, the obfuscated data provided by the device 100 can be de-obfuscated (e.g., unencrypted) at the remote location 170.

In some embodiments, the device 100 may communicate with remote computing devices 160 and/or 172 without the intermediation of the local computing device 150. For example, the vascular access device 100 may be connected via Wi-Fi or other wireless communications link to a network such as the Internet. In other embodiments, the device 100 may be in communication only with the local computing device 150, which in turn is in communication with remote computing devices 160 and/or 172.

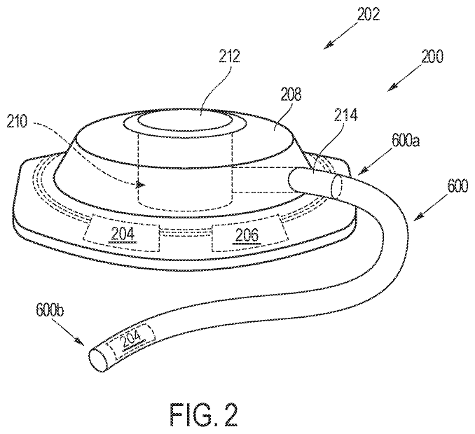

FIG. 2 shows an example of a vascular access device 200 (or “device 200”) configured for use with the systems of the present technology (including system 10). As shown in FIG. 2, the device 200 comprises a hub 202 and a catheter 600 configured to be permanently or detachably coupled to the hub 202. The device 200 further comprises a sensing element 204 carried by the hub 202 and/or catheter 600 and configured to obtain measurements indicative of a health of the patient and/or a performance of the device 200. As used herein, the term “sensing element” may refer to a single sensor or a plurality of discrete, separate sensors. The device 200 may further include an electronics component 206, such as a controller, configured to be communicatively coupled to the sensing element 204. In some embodiments, the sensing element 204 and/or electronics component 206 are carried by a printed circuit board (PCB). As discussed in greater detail below, the sensing element 204 can be configured to obtain data characterizing an identity parameter of the patient, a physiological parameter of the patient, a performance parameter of the device, a treatment parameter, and/or another parameter.

The hub 202 may comprise a housing 208, a fluid reservoir 210 contained within the housing 208, and a septum 212 adjacent the reservoir 210 that is configured to receive a needle therethrough for delivery of a fluid (such as a therapeutic or diagnostic agent) to the reservoir 210. The housing 208 may be made of a biocompatible plastic, metal, ceramic, medical grade silicone, or other material that provides sufficient rigidity and strength to prevent inadvertent needle puncture through the housing 208. The septum 212 can be, for example, a self-sealing membrane made of silicone or other deformable, self-sealing, biocompatible material.

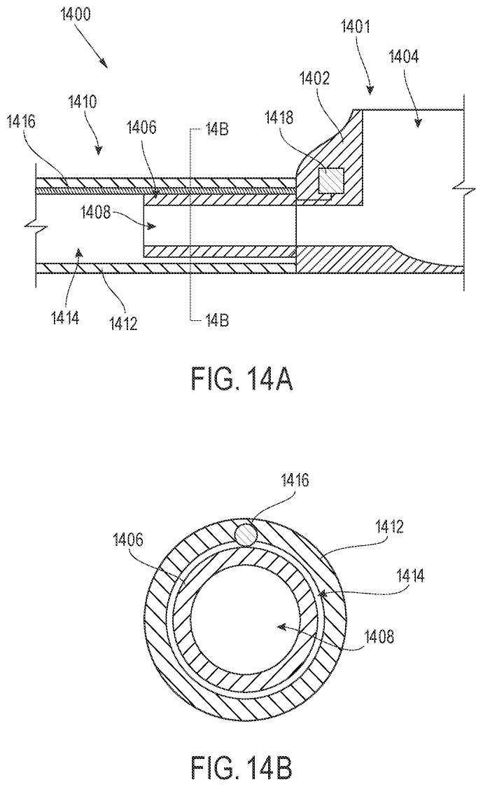

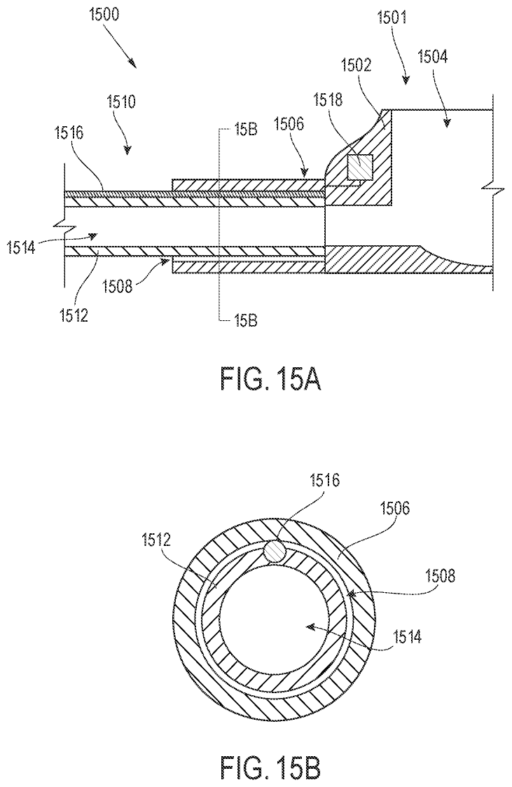

The catheter 600 can be configured to permanently or detachably couple to the hub 202 to be placed into fluid communication with the reservoir 210. For example, as shown in FIG. 2, the catheter 600 can have a proximal end portion 600a configured to mate with an outlet port 214 of the hub 202 (e.g., via a barb connector or other suitable mechanical connection) and a distal end portion 600b configured to be positioned within a blood vessel or a heart of a patient. Example connections between the catheter 600 and the hub 202 are discussed below with reference to FIGS. 14A-15B. The device 200 and/or system can comprise a single catheter 600 (as shown in FIG. 2) or multiple catheters 600. Moreover, the catheter 600 can define a single lumen or multiple lumens, as detailed herein.

As shown in FIG. 2, the device 200 can comprise one or more sensing elements 204 carried by the housing 208 and/or one or more sensing elements 204 carried by the catheter 600. Although two sensing elements 204 are illustrated for clarity in FIG. 2, in various embodiments, the device 200 may include zero, one, two, or more than two sensing elements 204 carried by the housing 208 and/or catheter 600. In some embodiments, one or more such sensing elements 204 may be disposed on separate structural components that are separated from the housing 208 and/or catheter 600. As described in greater detail herein, the device 200 can comprise one or more sensing elements 204 carried by the catheter 600, for example, to measure an operating parameter of the device 200, such as flow rate within the catheter 600, a pressure within the reservoir 210, etc. Additionally or alternatively, a sensing element 204 carried by the catheter can be configured to measure one or more physiological parameters of the patient, such as heart rate, respiratory rate, central venous pressure, temperature, a hematologic parameter (e.g., glucose, lactate, electrolytes, blood counts, etc.), and others.

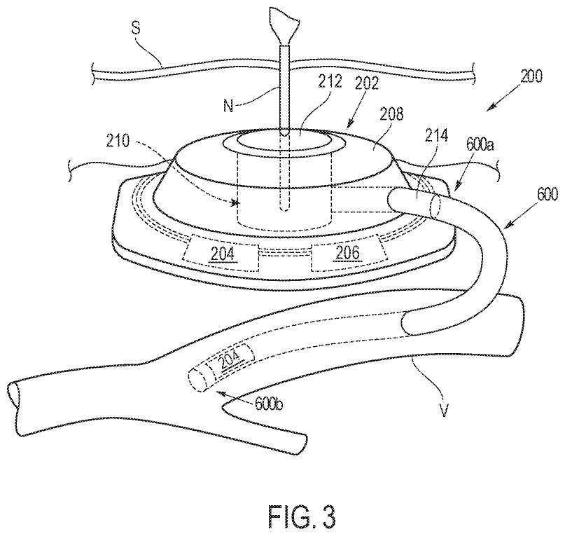

As shown in FIG. 3, the hub 202 can be implanted beneath the patient's skin S, and the catheter 600 can be inserted into a targeted cardiovascular location, such as a targeted blood vessel V. The blood vessel V, for example, can be an artery or a vein, such as the internal jugular vein or the subclavian vein. In operation, a clinician inserts a needle N (e.g., a non-coring or Huber-type needle) through the skin S, through the self-sealing septum 212, and into the fluid reservoir 210. To introduce fluid (e.g., medication, contrast agent, saline solution, etc.) into the patient's blood vessel V, the clinician may inject the fluid through the needle N, which then flows through the reservoir 210, the catheter 600, and into the vessel V. In some cases, the physician may inject fluid through the needle to fill the reservoir 210 for postponed delivery into the vessel V. To remove fluid from the vessel V (e.g., to aspirate blood from the vessel V for testing), the clinician can apply suction via the needle N, thereby withdrawing fluid (e.g., blood) from the vessel V into the catheter 600, into the fluid reservoir 210, and into the needle N. After aspiration or fluid delivery, the device 200 can be flushed and/or locked. A flushing fluid (e.g., saline solution, heparin solution, etc.) can be injected through the needle N, into the reservoir 210, and out of the catheter 600 such that the flushing fluid transports undesired material (e.g., residual medication, debris, etc.) out of the device 200. In some embodiments, the device 200 can be locked by delivering a volume of a locking fluid (e.g., a solution carrying at least one of sodium chloride, an anticoagulant agent, a thrombolytic agent, an antimicrobial agent, an antiseptic agent, or another suitable agent) to the catheter 600 such that the locking fluid remains in the catheter lumen and prevents or limits ingress of blood into the catheter lumen or occlusion of the catheter lumen. When the procedure is completed, the clinician removes the needle N, the self-sealing septum 212 resumes a closed configuration, and the device 200 may remain in place beneath the patient's skin S.

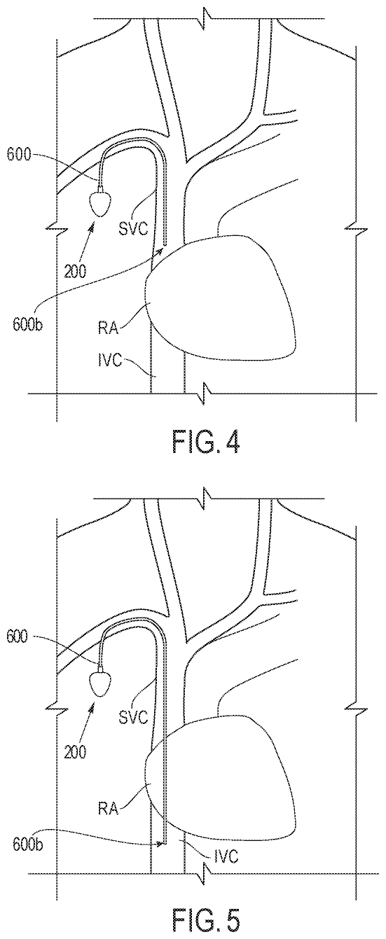

The catheter 600 can have different lengths depending on the desired positioning of the distal end portion 600b of the catheter 600, such as within the superior vena cava SVC, the right atrium RA, the inferior vena cava IVC, or another suitable cardiovascular location. FIG. 4, for example, shows the device 200 implanted in a patient with the distal end portion 600b of the catheter 600 positioned within the patient's superior vena cava SVC. Delivery of medication into a large diameter vein with a large volume of blood, such as the superior vena cava SVC, can be beneficial as it prevents or limits damage to the vessel wall that can be caused by delivery of certain medications (e.g., hazardous chemotherapeutics, etc.) in close proximity to the vessel wall and/or towards the vessel wall. Moreover, when the distal end portion 600b of the catheter 600 is positioned within the superior vena cava SVC, fluid flows through the catheter 600 and out of the distal end portion 600b of the catheter 600 along a path oriented in the same direction as the flow of blood within the vessel, which can permit or facilitate fluid delivery at a low pressure and/or flow rate.

In some embodiments, for example as shown in FIG. 5, the device 200 and/or catheter 600 can be configured such that, when implanted, the distal end portion 600b of the catheter 600 is positioned distal of the superior vena cava SVC, such as within the right atrium RA or the inferior vena cava IVC. A catheter with a greater length (e.g., catheter 600 shown in FIG. 5 as compared to catheter 600 shown in FIG. 4) can have certain advantages, such as the ability to accommodate more sensing elements. Additionally or alternatively, the catheter 600 may comprise one or more sensing elements 204 positioned along its length relative to the distal tip such that placement of the distal end portion 600b within the right atrium RA and/or inferior vena cava IVC locates at least one of the sensing elements 204 within the right atrium RA and/or inferior vena cava IVC. Such positioning can provide higher fidelity physiologic measurements. For example, a sensing element 204 configured to detect an electrical signal of the patient's heart can detect the electrical signal with greater accuracy if positioned within the right atrium RA instead of the superior vena cava SVC. As shown in FIG. 5, when the distal end portion 600b is positioned within the right atrium RA or inferior vena cava IVC, the distal end portion 600b and/or other portions of the catheter 600 distal of the junction between the superior vena cava SVC and the right atrium RA can have a substantially straight configuration. This straighter configuration may be advantageous for the performance of certain sensing elements carried by the catheter 600. For example, in one embodiment, electrodes for monitoring cardiac electrical activity placed on the straight distal portion of the catheter 600 will detect electrical conduction through the inferior wall of the heart. The straighter configuration can also prevent or limit delivery of potentially hazardous medications or other fluids into a vessel wall. A straight configuration will also limit repeated traumatic erosion of the vessel wall that is possible with other catheter configurations. Still, other configurations are possible in which the catheter 600 and/or the distal end portion 600b of the catheter 600 are not positioned within the superior vena cava SVC or the inferior vena cava IVC.

Referring back to FIGS. 2 and 3, an electronic component 206 comprising a controller may include one or more processors, software components, and/or memory (not shown). In some examples, the one or more processors include one or more computing components configured to process measurements received from the sensing element 204 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensing element 204 to obtain one or more measurements, such as data characterizing a physiological parameter of the patient, data characterizing a performance of the catheter 600, data characterizing a performance of the hub 202, data characterizing a type of needle N inserted into the reservoir 210, etc. In another example, the functions may involve processing the data to determine one or more parameters and/or provide an indication to the patient and/or clinician of a health of the patient, a performance of the device, and/or a status of a treatment. For example, the functions may involve providing an indication to the patient and/or clinician of one or more symptoms or medical conditions associated with determined physiological parameters.

The controller may also include a data communications unit configured to securely transmit data between the device 200 and external computing devices (e.g., local computing device 150, remote computing devices 160 and 172, etc.). In some embodiments, the controller includes a localization unit configured to emit a localization signal (e.g., lights that transilluminate a patient's skin, vibration, a magnetic field, etc.) to aid a clinician in localizing the device 200 when implanted within a patient. The controller can also include a wireless charging unit (such as a coil) configured to recharge a battery (not shown) of the device 200 when in the presence of an interrogation device (e.g., local device 150 or another suitable device).

The system 10 may be configured to continuously and/or periodically obtain measurements via the sensing element 204. The sensing element 204 may be carried by the housing 208 and/or the catheter 600, and/or may include a sensing component separate from the housing 208 and catheter 600 but physically or wirelessly communicatively coupled to the housing 208 and/or catheter 600. The sensing element 204 may be implanted at the same location as the device 200 or at a different location, or may be positioned on the patient at an exterior location (e.g., on the patient's skin). The sensing element 204 may be permanently coupled to the device 200, or may be configured to temporarily couple to the device 200.

In some embodiments, the sensing element 204 is built into the housing 208 and/or the catheter 600 such that only a portion of the sensing element 204 is exposed to the local physiological environment when the device 200 is implanted. For example, the sensing element 204 may comprise one or more electrodes having an external portion positioned at an exterior surface of the housing 208 and/or the catheter 600 and an internal portion positioned within the housing 208 and/or the catheter 600 and, optionally, wired to the controller. In some embodiments, the sensing element 204 may comprise one or more electrodes having an internal portion positioned at an interior surface of the housing 208 at the interface with the port reservoir 210 or junction of the reservoir 210 and the catheter 600, or extending into the catheter 600.

In some embodiments the sensing element 204 may be completely contained within the housing 208 and/or the catheter 600. For example, the sensing element 204 may comprise one or more pulse oximeters enclosed by the housing 208 and/or the catheter 600 and positioned adjacent a window in the housing 208 and/or the catheter 600 through which light emitted from the pulse oximeter may pass to an external location, and back through which light reflected from the external location may pass for detection by a photodiode of the pulse oximeter. In such embodiments the window may be, for example, a sapphire window that is brazed into place within an exterior wall of the housing 208 and/or the catheter 600.

The sensing element 204 may comprise at least one sensor completely enclosed by the housing 208 and/or the catheter 600 and at least one sensor that is partially or completely positioned at an external location, whether directly on the housing 208 and/or catheter 600 or separated from the housing 208 and/or catheter 600 (but still physically coupled to the housing 208 and/or catheter 600 via a wired connection, for example). In some embodiments, at least a portion of the sensing element 204 is positioned at and/or exposed to an interior region of the reservoir 210. In some embodiments, at least a portion of the sensing element 204 is positioned at and/or exposed to a lumen of the catheter 600.

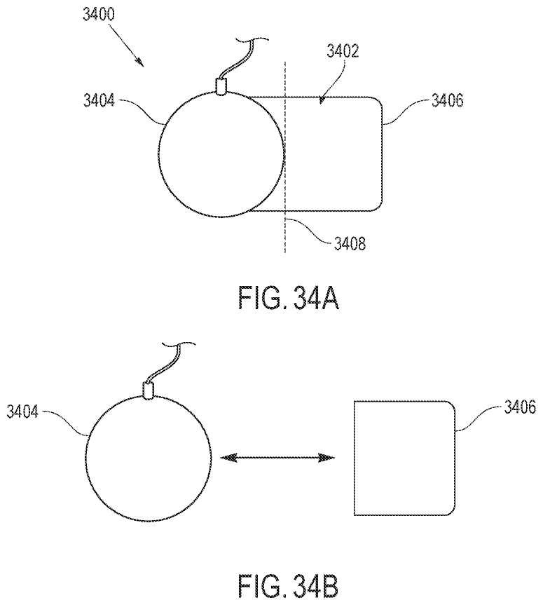

The sensing element 204 and/or electronic component 206 can be mounted to one or both sides of a PCB. The hub 202 and/or the catheter 600 can carry a PCB mounting one sensing element 204, a PCB mounting one electronic component 206, a PCB mounting multiple sensing elements 204 and/or a PCB mounting multiple electronic components 206. Although FIGS. 2 and 3 depict the housing 208 being substantially cylindrical with the sensing element 204 and electronic component 206 distributed around a circumference of the housing 208 and reservoir 210, other configurations are possible. For example, the hub 202 can comprise a reservoir portion and an electronics portion that is offset from the reservoir portion (see, for example, FIGS. 34A and 34B). In these and other embodiments, the electronics portion can comprise one or multiple PCBs mounting the sensing element 204 and/or electronic component 206.

In some embodiments, the sensing element 204 may include a separate controller (not shown) that comprises one or more processors and/or software components. In such embodiments, the sensing element 204 may process at least some of the measurements to determine one or more parameters, and then transmit those parameters to the controller of the device 200 (with or without the underlying data). In some examples, the sensing element 204 may only partially process at least some of the measurements before transmitting the data to the controller. In such embodiments, the controller may further process the received data to determine one or more parameters. The local computing device 150 and/or the remote computing devices 160, 172 may also process some or all of the measurements obtained by the sensing element 204 and/or parameters determined by the sensing element 204 and/or the controller.

According to some aspects of the technology, the sensing element 204 may include memory. The memory may be a non-transitory computer-readable medium configured to permanently and/or temporarily store the measurements obtained by the sensing element 204. In those embodiments where the sensing element 204 includes its own processor(s), the memory may be a tangible, non-transitory computer-readable medium configured to store instructions executable by the processor(s).

In some embodiments, the sensing element(s) 204 and/or controller may be configured to detect, identify, monitor, and/or communicate information by electromagnetic, acoustic, motion, optical, thermal, or biochemical sensing elements or means. The sensing element(s) 204 may include, for example, one or more temperature sensing elements (e.g., one or more thermocouples, one or more digital temperature sensors, one or more thermistors or other type of resistance temperature detector, etc.), one or more impedance sensing elements (e.g., one or more electrodes), one or more pressure sensing elements, one or more optical sensing elements, one or more flow sensing elements (e.g., a Doppler velocity sensing element, an ultrasonic flow meter, etc.), one or more ultrasonic sensing elements, one or more photoplethysmography (PPG) sensing elements (e.g., pulse oximeters, etc.), one or more chemical sensing elements, one or more movement sensing elements (e.g., one or more accelerometers), one or more pH sensing elements, an electrocardiogram (“ECG” or “EKG”) unit, one or more electrochemical sensing elements, one or more hemodynamic sensing elements, and/or other suitable sensing devices.

The sensing element 204 may comprise one or more electromagnetic sensing elements configured to measure and/or detect, for example, impedance, voltage, current, or magnetic field sensing capability with a wire, wires, wire bundle, magnetic node, and/or array of nodes. The sensing element 204 may comprise one or more acoustic sensing elements configured to measure and/or detect, for example, sound frequency, within human auditory range or below or above frequencies of human auditory range, beat or pulse pattern, tonal pitch melody, and/or song. The sensing element 204 may comprise one or more motion sensing elements configured to measure and/or detect, for example, vibration, movement pulse, pattern or rhythm of movement, intensity of movement, and/or speed of movement. Motion communication may occur by a recognizable response to a signal. This response may be by vibration, pulse, movement pattern, direction, acceleration, or rate of movement. Motion communication may also be by lack of response, in which case a physical signal, vibration, or bump to the environment yields a motion response in the surrounding tissue that can be distinguished from the motion response of the sensing element 204. Motion communication may also be by characteristic input signal and responding resonance. The sensing element 204 may comprise one or more optical sensing elements which may include, for example, illuminating light wavelength, light intensity, on/off light pulse frequency, on/off light pulse pattern, passive glow or active glow when illuminated with special light such as UV or “black light”, or display of recognizable shapes or characters. It also includes characterization by spectroscopy, interferometry, response to infrared illumination, and/or optical coherence tomography. The sensing element 204 may comprise one or more thermal sensing elements configured to measure and/or detect, for example, device 200 temperature relative to surrounding environment, the temperature of the device 200 (or portion thereof), the temperature of the environment surrounding the device 200 and/or sensing element 204, or differential rate of the device temperature change relative to surroundings when the device environment is heated or cooled by external means. The sensing element 204 may comprise one or more biochemical devices which may include, for example, the use of a catheter, a tubule, wicking paper, or wicking fiber to enable micro-fluidic transport of bodily fluid for sensing of protein, RNA, DNA, antigen, and/or virus with a micro-array chip.

In some aspects of the technology, the controller and/or sensing element 204 may be configured to detect and/or measure the concentration of blood constituents, such as sodium, potassium, chloride, bicarbonate, creatinine, blood urea nitrogen, calcium, magnesium, and phosphorus. The system 10 and/or the sensing element 204 may be configured to evaluate liver function (e.g., by evaluation and/or detection of AST, ALT, alkaline phosphatase, gamma glutamyl transferase, troponin, etc.), heart function (e.g., by evaluation and/or detection of troponin), coagulation (e.g., via determination of prothrombin time (PT), partial thromboplastin time (PTT), and international normalized ratio (INR)), and/or blood counts (e.g., hemoglobin or hematocrit, white blood cell levels with differential, and platelets). In some embodiments, the system 10 and/or the sensing element 204 may be configured to detect and/or measure circulating tumor cells, circulating tumor DNA, circulating RNA, multigene sequencing of germ line or tumor DNA, markers of inflammation such as cytokines, C reactive protein, erythrocyte sedimentation rate, tumor markers (PSA, beta-HCG, AFP, LDH, CA 125, CA 19-9, CEA, etc.), and others.

As previously mentioned, the system 10 may determine one or more physiological parameters based on data obtained by the sensing element 204 and/or one or more other physiological parameter(s). For example, the system 10 may be configured to determine physiological parameters such as heart rate, temperature, blood pressure (e.g., systolic blood pressure, diastolic blood pressure, mean arterial blood pressure), cardiac output, ejection fraction, pulmonary artery pressure, pulmonary capillary wedge pressure, left atrial pressure, blood flow rate, blood velocity, pulse wave speed, volumetric flow rate, reflected pressure wave amplitude, augmentation index, flow reserve, resistance reserve, resistive index, capacitance reserve, hematocrit, heart rhythm, electrocardiogram (ECG) tracings, body fat percentage, activity level, body movement, falls, gait analysis, seizure activity, blood glucose levels, drug/medication levels, blood gas constituents and blood gas levels (e.g., oxygen, carbon dioxide, etc.), lactate levels, hormone levels (such as cortisol, thyroid hormone (T4, T3, free T4, free T3), TSH, ACTH, parathyroid hormone), medication concentration in the blood, pharmacokinetic and pharmacodynamic data, and/or any correlates and/or derivatives of the foregoing measurements and parameters (e.g., raw data values, including voltages and/or other directly measured values). In some embodiments, data obtained by the sensing element 204 can be utilized or characterized as a physiological parameter without any additional processing by the system 10.

Additionally or alternatively, the sensing element 204 can be configured to obtain data characterizing a parameter associated with performance of the device, treatment of the patient, etc. For example, the sensing element 204 can be configured to obtain data characterizing a flow rate parameter within the catheter 600 and/or the reservoir 210, a pressure within the catheter 600 and/or the reservoir 210, a temperature of one or more portions of the device 200, a presence and/or position of an object (e.g., a needle, fluid, a clot, etc.) within the reservoir 210 and/or catheter 600, information encoded by machine-readable indicia, etc.

The system 10 may also determine and/or monitor derivatives of any of the foregoing parameters (e.g., physiological parameters, device performance parameters, treatment parameters, identity parameters, etc.), such as a rate of change of a particular parameter, a change in a particular parameter over a particular time frame, etc. As but a few examples, the system 10 may be configured to determine a temperature over a specified time, a maximum temperature, a maximum average temperature, a minimum temperature, a temperature at a predetermined or calculated time relative to a predetermined or calculated temperature, an average temperature over a specified time, a maximum blood flow, a minimum blood flow, a blood flow at a predetermined or calculated time relative to a predetermined or calculated blood flow, an average blood flow over time, a maximum impedance, a minimum impedance, an impedance at a predetermined or calculated time relative to a predetermined or calculated impedance, a change in impedance over a specified time, a change in impedance relative to a change in temperature over a specified time, a change in heart rate over time, a change in respiratory rate over time, activity level over a specified time and/or at a specified time of day, and other suitable derivatives.

Data may be obtained continuously or periodically at one or more predetermined times, ranges of times, calculated times, and/or times when or relative to when a measured event occurs. Likewise, parameters (physiological or otherwise) may be determined continuously or periodically at one or more predetermined times, ranges of times, calculated times, and/or times when or relative to when a measured event occurs.

Based on the determined parameters, the system 10 of the present technology can be configured to provide an indication of the patient's health, the performance and/or health of device, and/or the status of a treatment to the patient and/or a clinician. For example, the controller may compare one or more physiological parameters to a predetermined threshold or range and, based on the comparison, provide an indication of the patient's health. If the determined physiological parameter(s) is above or below the predetermined threshold or outside of the predetermined range, the system 10 may provide an indication that the patient is at risk of, or has already developed, a medical condition characterized by symptoms associated with the determined physiological parameters. As used herein, a “predetermined range” refers to a set range of values, and “outside of a/the predetermined range” refers to (a) a measured or calculated range of values that only partially overlap the predetermined range or do not overlap any portion of a predetermined range of values. As used herein, a “predetermined threshold” refers to a single value or range of values, and a parameter that is “outside” of “a predetermined threshold” refers to a situation where the parameter is (a) a measured or calculated value that exceeds or fails to meet a predetermined value, (b) a measured or calculated value that falls outside of a predetermined range of values, (c) a measured or calculated range of values that only partially overlaps a predetermined range of values or does not overlap any portion of a predetermined range of values, or (d) a measured or calculated range of values where none of the values overlap with a predetermined value.

Predetermined parameter thresholds and/or ranges can be empirically determined to create a look-up table. Look-up table values can be empirically determined, for example, based on clinical studies and/or known healthy or normal values or ranges of values. The predetermined threshold may additionally or alternatively be based on a particular patient's baseline physiological parameters, a particular device's baseline performance parameters, etc. In some embodiments, the system 10 can be configured to determine the predetermined threshold and/or range based on data collected by the device to determine a patient's normal or baseline parameter or range of parameters. In some cases, a patient's baseline parameter or range of parameters may differ from known normal parameters/ranges of parameters. For example, a patient with anemia may have a lower baseline blood oxygenation saturation than a non-anemic patient. Accordingly, a predetermined threshold for blood oxygenation saturation that indicates that the patient's blood oxygenation saturation is abnormal and/or indicative of a medical condition may be lower for the anemic patient than for a non-anemic patient.

In some embodiments, the controller may be configured to detect a pattern of measurements (of a single parameter or a combination of parameters) indicative of a health condition. In such embodiments, the individual measurements may not fall outside of a given “normal” range yet, when considered together, can still indicate a change in health status. For example, the controller may be configured to identify patterns of change in temperature, heart rate, and activity that are associated with infection, even if all three are still within a “normal” range.

Medical conditions detected and/or indicated by the system 10 may include, for example, sepsis, pulmonary embolism, metastatic spinal cord compression, anemia, dehydration/volume depletion, vomiting, pneumonia, congestive heart failure, performance status, arrythmia, neutropenic fever, acute myocardial infarction, pain, opioid toxicity, nicotine or other drug addiction or dependency, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, volume overload, hyperuricemia, and/or seizure. Any of the systems and/or devices disclosed herein may be used to monitor a patient for any of the foregoing medical conditions.

The system 10 can be configured to provide notifications to a patient and/or a clinician. For example, the device 100 and/or an external computing device (e.g., a smartphone, a PC, etc.) can be configured to provide a notification to the patient if a battery carried by the device 100 is low and needs to be recharged. The system 10 may be configured to provide a notification to a patient and/or clinician if it determines that the device is occluded, overheating, has lost wireless communication, is infected or colonized, or is otherwise malfunctioning. In some embodiments, the device 100 includes a notification unit configured to provide notifications to the patient without the need for an external computing device. The notification unit can include a speaker, a light, a vibration element, or another means for providing audible, visual, haptic, and/or tactile notifications to the patient. As an example, the notification unit can comprise a vibration element configured to vibrate if the patient's blood glucose has fallen outside of predetermined healthy or normal range to indicate to the patient that intervention should be taken to regulate their blood glucose.

In some embodiments, one or more parameters of a notification provided by a notification unit can be based on a type of information to be communicated to a patient. For example, a high frequency, high amplitude vibration can communicate to the patient that their blood pressure is higher than a predetermined threshold, while a low frequency, low amplitude vibration can communicate to the patient that their blood pressure is lower than a predetermined threshold. Further, various notification modalities and/or parameters can be used alone or in combination to communicate specific information to the patient. For example, the notification unit can include an LED light and a speaker. If the system 10 determines that the patient is experiencing ventricular fibrillation, the LED light can emit red light and the speaker can emit an audible notification instructing listeners to call an ambulance. In some embodiments, the LED light can emit a light of a specific color to indicate that the device 100 needs to be recharged.

According to various embodiments of the present technology, the device 100 can comprise a voice recognition unit configured to obtain audio data. The voice recognition unit can include a microphone, such as any of the microphones described herein, and a controller communicatively coupled to the microphone. In some embodiments, the voice recognition unit or one or more portions thereof is communicatively coupled to the sensing element 102 or another electronic component carried by the device 100. In some embodiments, the voice recognition unit comprises a microphone communicatively coupled to a separate controller carried by device 100 and/or an external controller.

The voice recognition unit can be configured to obtain audio data and, in some embodiments, transmit the audio data to the controller and/or sensing element 102. In some embodiments, the controller can be configured to cause the device 100 to perform a function or action, based at least in part on the audio data. For example, a person (e.g., a patient, a clinician, etc.) can provide an audible instruction for the device 100 to obtain data characterizing a temperature of the person. Providing the audible instruction can include saying out loud “Device, take the patient's temperature.” The voice recognition unit can obtain audio data characterizing the sound waves produced by the person providing the audible instruction. The audio data can be transmitted to a controller, which can process the audio data and, based on the audio data, cause the sensing element 102 carried by the device 100 to obtain data characterizing a temperature of the patient. In some embodiments, the controller can process the temperature data and/or cause the notification unit to provide a notification communicating the patient's temperature. In another example, a person can provide an audible instruction for the device 100 to turn on, wake up, exit a lower-power mode, or otherwise activate.

As previously noted, a vascular access device of the present technology can include a catheter carrying a sensing element, whose positioning can facilitate and/or enable collection of specific data by the sensing element. For example, a photoelectric sensing element configured to emit light into a lumen of the catheter and detect light reflected by the contents of the lumen can be carried by the catheter such that the sensor is exposed to the lumen of the catheter (e.g., directly, via a window, etc.) to permit the intended emission and detection of light. A sensing element carried by the catheter can be configured to obtain data characterizing a performance of the catheter. For example, a catheter can become obstructed if a blood clot forms in, on, or at the distal end of the catheter, which can hinder or prevent fluid delivery and/or aspiration through the device. A catheter of the present technology can carry a sensing element configured to measure a flow rate within the catheter, a pressure within the catheter, or another parameter such that the system is configured to evaluate a performance and/or functioning of the catheter. Additionally or alternatively, a sensing element carried by the catheter can be configured to be positioned distal of a hub of the vascular access device, which can enhance performance of the sensing element. For example, a central venous pressure measurement can be obtained with a greater accuracy by obtaining the measurement from a sensing element positioned in one of the venae cavae near the right atrium of the heart instead of a sensing element positioned at a proximal location in the superior vena cava.

A sensing element carried by a catheter can be configured to communicate with one or more data communications units and/or one or more remote computing devices. In some embodiments, a sensing element carried by a catheter can be configured to communicate with one or more electronic components (e.g., one or more additional sensing elements, one or more controllers, etc.) carried by the hub of the vascular access device. As described in greater detail below, a sensing element carried by a catheter can be configured to communicate via a wired connection or a wireless connection.

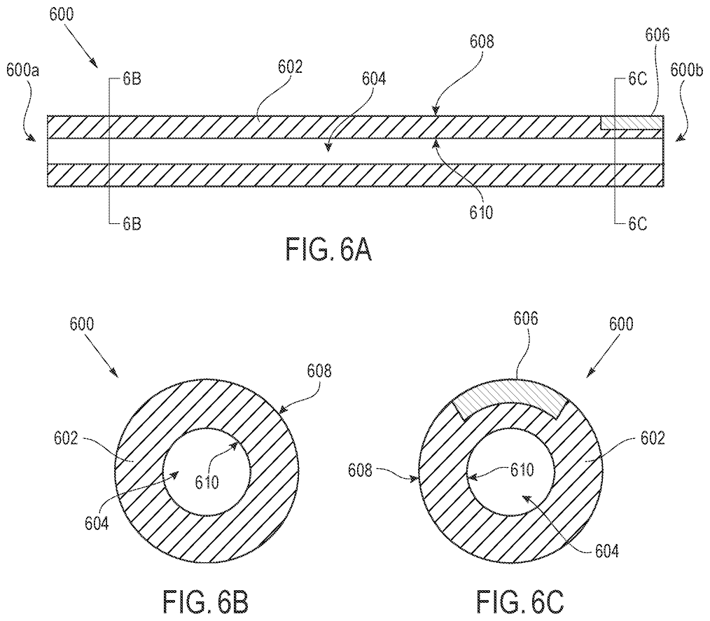

FIG. 6A is a side cross-sectional view of a portion of a catheter 600 configured in accordance with several embodiments of the present technology, and FIGS. 6B and 6C are axial cross-sectional views of the catheter 600 taken along lines 6B-6B and 6C-6C, respectively. The catheter 600 has a proximal end portion 600a, a distal end portion 600b, and a sidewall 602 defining a lumen 604. The catheter 600 can comprise an abluminal surface 608 and a luminal surface 610. The catheter 600 further comprises a sensing element 606 configured to communicate wirelessly with other electronics components (such as other sensing elements, a controller, etc.), which may be located on the catheter 600, at a corresponding hub (not shown), or at an extracorporeal location. For example, the sensing element 606 can be configured to communicate with other electronic components via near-field communication (NFC), infrared wireless, Bluetooth, ZigBee, Wi-Fi, inductive coupling, capacitive coupling, or any other suitable wireless communication link. The sensing element 606 can be battery-powered and/or configured to be charged wirelessly.

In FIG. 6A, the catheter 600 includes a single sensing element 606 positioned at the distal end portion 600b of the catheter 600. Locating the sensing element 606 at the distal end portion 600b can be beneficial for detecting certain physiological measurements (e.g., central venous pressure, core temperature, etc.) and/or increasing a distance between the sensing element 606 and another sensing element or electronic component (e.g., to increase an electrical potential measured between two electrodes carried by the catheter 600, etc.). As best shown in FIG. 6C, the sensing element 606 can be positioned within a thickness of the sidewall 602 such that a portion of the sensing element 606 is exposed at the exterior of the sidewall 602. In some embodiments, it may be advantageous and/or necessary for a portion of the sensing element 606 to be exposed to the exterior of the sidewall 602 to permit a physical, electrical, or chemical interaction between the sensing element 606 and the environment surrounding the catheter 600. For example, if the sensing element 606 comprises an electrode, the sensing element 606 may need to be at least partially exposed the environment surrounding the catheter 600 to establish electrical contact with the environment. It will be appreciated that other numbers, configurations, and locations of sensing elements 606 are possible. For example, the catheter 600 can comprise a plurality of sensing elements 606 (e.g., two sensing elements 606, three sensing elements 606, four sensing elements 606, etc.), and any of the sensing elements can be positioned at any longitudinal, circumferential, and radial position along the sidewall, as described herein.

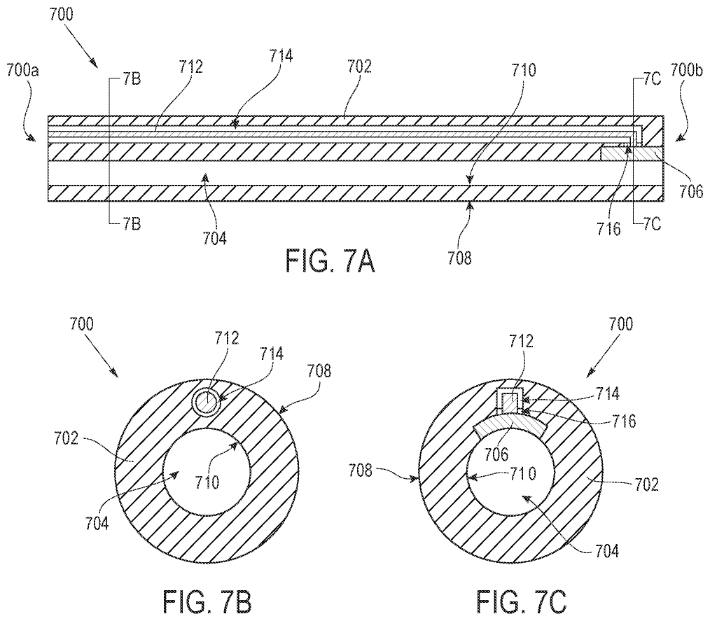

According to several embodiments, the catheter can include a sensing element configured to charge and/or communicate via a wired connection. FIGS. 7A-7C depict one example of such a catheter. FIG. 7A is a side cross-sectional view of a portion of a catheter 700 and FIGS. 7B and 7C are axial cross-sectional views of the catheter 700 taken along lines 7B-7B and 7C-7C, respectively. The catheter 700 has a proximal end portion 700a, a distal end portion 700b, and a sidewall 702 defining a lumen 704. As shown in FIG. 7A, the catheter 700 can carry a sensing element 706 near and/or at the distal end portion 700b of the catheter 700. Still other numbers and positions of sensing elements 706 are possible as described herein.

As shown in FIGS. 7A-7C, the catheter 700 can carry a conductive element 712 having a distal end electrically coupled to the sensing element 706. A proximal end of the conductive element 712 can be electrically coupled to a power source, another sensing element, a controller, or another suitable electronic component. For example, the proximal end of the conductive element 712 can be electrically coupled to a controller carried by a hub of the vascular access device. In some embodiments, the conductive element 712 can be positioned within a channel 714 defined by the sidewall 702 of the catheter 700. The conductive element 712 can extend through an aperture 716 from the channel 714 to the sensing element 706. According to various embodiments, the conductive element 712 can be positioned within the lumen 704 of the catheter 700, on the abluminal surface 708 of the catheter 700, and/or on the luminal surface 710 of the catheter 700. In some embodiments, the conductive element 712 can be coextruded with the sidewall 702 of the catheter 700.

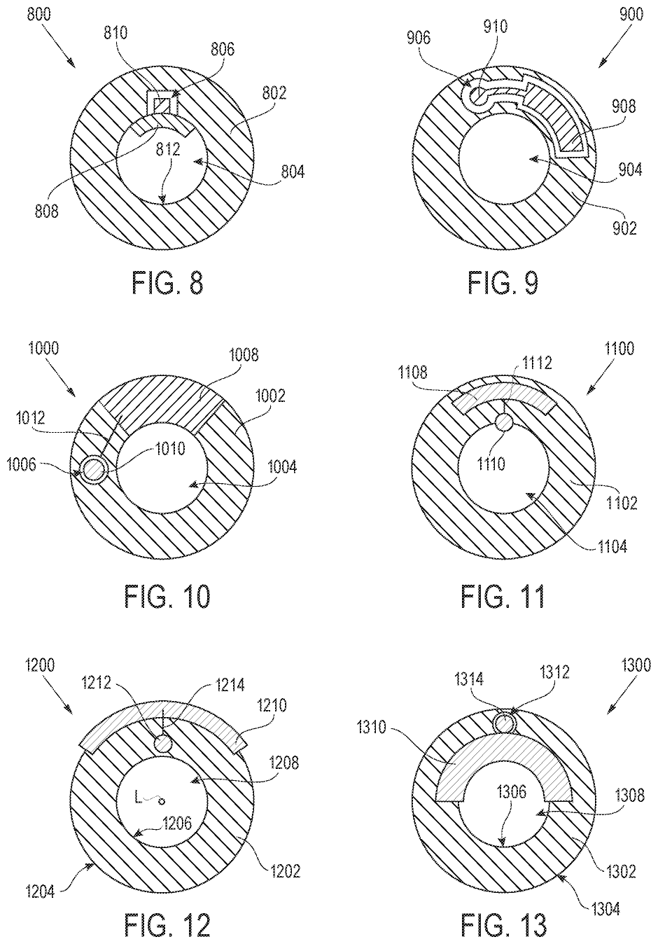

FIGS. 8-13 are example axial cross-sectional views of catheters carrying a sensing element and a conductive element. Any of the catheters of the present technology can include any of the sensing elements and/or any of the conductive elements depicted in FIGS. 8-13 in any suitable combination. For example, the catheter 600 can include sensing element 808 in addition to sensing element 204 and/or in place of sensing element 204.

As shown in FIG. 8, in some embodiments a catheter 800 can comprise a sidewall 802 defining a lumen 804 and a channel 806. A sensing element 808 can be positioned within the lumen 804 and a conductive element 810 can be positioned within the channel 806. A luminal surface 812 of the sidewall 802 can have an opening to permit electrical coupling and/or communication between the sensing element 808 and the conductive element 810.

FIG. 9 depicts a catheter 900 comprising a sidewall 902 defining a lumen 904 and a channel 906. In contrast to the configuration depicted in FIG. 8, a sensing element 908 and a conductive element 910 carried by the catheter 900 can both be positioned within the channel 906. The sensing element 908 and/or the conductive element 910 can be wholly contained within the channel 906 (see FIG. 9) and/or one or more portions of the sensing element 908 and/or the conductive element 910 can be positioned outside of the channel 906. As previously noted, the conductive element 910 can extend along a longitudinal axis of the catheter. As shown in FIG. 9, in some embodiments one or more portions of the conductive element 910 extends circumferentially and/or radially toward the sensing element 908.

As shown in FIG. 10, in some embodiments a catheter 1000 comprises a sidewall 1002 defining a lumen 1004 and a channel 1006, a sensing element 1008 embedded in the sidewall 1002, and a conductive element 1010 positioned in the channel 1006. The channel 1006 can be spaced apart from the sensing element 1008, as shown in FIG. 10. In these and other embodiments, a conductive lead 1012 can extend from the conductive element 1010 to the sensing element 1008 through the sidewall 1002 of the catheter 1000.

Although FIG. 10 depicts the conductive element 1010 positioned within the channel 1006, which is positioned entirely within the sidewall 1002 of the catheter 1000, other configurations are possible. For example, FIG. 11 depicts a catheter 1100 comprising a sidewall 1102 defining a lumen 1104 of the catheter 1100, but a sensing element 1108 and a conductive element 1110 electrically coupled to the sensing element 1108 are positioned within the sidewall 1102, rather than within a channel. As shown in FIG. 11, in some embodiments at least a portion of the conductive element 1110 is positioned in the lumen 1104 of the catheter 1100. The sensing element 1108 and conductive element 1110 can be radially spaced apart (see FIG. 11), circumferentially spaced apart, and/or longitudinally spaced apart. In these and other embodiments, the catheter 1100 can include a conductive lead 1112 having a first end electrically coupled to the sensing element 1108 and a second end electrically coupled to the conductive element 1110 to facilitate electrical communication between the sensing element 1108 and the conductive element 1110.

FIG. 12 depicts a catheter 1200 can comprise a sidewall 1202 having an abluminal surface 1204 and a luminal surface 1206, which defines a lumen 1208 of the catheter 1200, and a sensing element 1210 carried by the catheter 1200. In some embodiments, for example as shown in FIG. 11, the sensing element 1108 can be entirely encased within the sidewall 1102 of the catheter 1100. However, as shown in FIG. 12, in some embodiments at least a portion of the sensing element 1210 is spaced apart from a central longitudinal axis L of the catheter 1200 by a greater radial distance than the abluminal surface 1204 of the catheter. The sensing element 1210 can be positioned partially within the sidewall 1202 of the catheter 1200 (see FIG. 12) or can be positioned on the abluminal surface 1204. The catheter 1200 can include a conductive element 1212 and/or a conductive lead 1214 electrically coupling the conductive element 1212 to the sensing element 1210.

FIG. 13 depicts a catheter 1300 comprising a sidewall 1302 having an abluminal surface 1304 and a luminal surface 1306, which defines a lumen 1308 of the catheter 6000. A sensing element 1310 can be carried by the catheter 1300. At least a portion of the sensing element 1310 can be spaced apart from a central longitudinal axis L of the catheter 1300 by a smaller radial distance than the luminal surface 1306 of the catheter 1300. The sensing element 1310 can be positioned partially within the sidewall 1302 of the catheter 1300 (see FIG. 13) or the sensing element 1310 can be positioned on the luminal surface 1306 of the catheter 1300. The sidewall 1302 can define a channel 1312, and a conductive element 1314 can be positioned within the channel 1312. As shown in FIG. 13, in some embodiments the channel 1312 extends at least partially through the abluminal surface 1304 of the catheter 1300 such that the channel is open to the environment surrounding the catheter 1300. Such configuration can enable or facilitate electrical coupling between the conductive element 1314 and a hub or other external device. Additionally or alternatively, one or more portions of the channel 1312 adjacent to the sensing element 1310 can be open to facilitate or enable electrical coupling between the sensing element 1310 and the conductive element 1314.

As previously described, and as shown in FIGS. 14A and 14B, a vascular access device 1400 in accordance with several embodiments of the present technology can comprise a hub 1401 including a housing 1402 defining a reservoir 1404 and forming an outlet port 1406. A lumen 1408 of the outlet port 1406 can be in fluidic communication with the reservoir 1404. A proximal end portion 1410a of a catheter 1410 having a sidewall 1412 defining a lumen 1414 can be secured to the outlet port 1406 such that the lumen 1408 of the outlet port 1406 is in fluidic communication with the lumen 1414 of the catheter 1410. As previously noted, the catheter 1410 can include an electronic component (not shown in FIGS. 14A and 14B), such as a sensor, a controller, a memory, a data communications unit, a power source, etc. In some embodiments, the catheter 1410 can include a conductive element 1416 configured to be electrically coupled to the electronic component carried by the catheter 1410. Additionally or alternatively, the housing 1402 and/or reservoir 1404 can include an electronic component 1418 (e.g., a sensing element, a controller, a memory, a power source, a data communications unit, etc.).

The catheter 1410 can be configured to be secured to the outlet port 1406 such that the conductive element 1416 is, either directly or indirectly, electrically coupled to the electronic component 1418 carried by the housing 1402. For example, the outlet port 1406 can comprise an electrically conductive material. The entire outlet port 1406 can comprise the electrically conductive material or the outlet port 1406 can comprise distinct regions of electrically conductive material. As shown in FIGS. 14A and 14B, the outlet port 1406 can be positioned within the lumen 1414 of the catheter 1410 such that the conductive element 1416 carried by the catheter 1410 and the outlet port 1406 are in electrical communication. In some embodiments, the conductive element 1416 or a portion thereof can be exposed to the lumen 1414 of the catheter 1410 such that, when the outlet port 1406 is positioned within the lumen 1414 of the catheter 1410, the conductive element 1416 can contact or otherwise electrically communicate with an abluminal surface of the outlet port 1406.

FIGS. 15A and 15B depict a vascular access device 1500 (also “device 1500”), which can be similar to the vascular access device 1400 except as detailed below. The device 1500 can comprise a hub 1501 including a housing 1502 defining a reservoir 1504 and forming an outlet port 1506. A lumen 1508 of the outlet port 1506 can be in fluidic communication with the reservoir 1504. A proximal end portion 1510a of a catheter 1510 having a sidewall 1512 defining a lumen 1514 can be secured to the outlet port 1506 such that the lumen 1508 of the outlet port 1506 is in fluidic communication with the lumen 1514 of the catheter 1510. As previously noted, the catheter 1510 can include an electronic component (not shown in FIGS. 15A and 15B), such as, but not limited to, a sensing element. In some embodiments, the catheter 1510 includes a conductive element 1516 electrically coupled to the electronic component. In some embodiments, the catheter 1510 is configured to be positioned within the lumen 1508 of the outlet port 1506 such that the conductive element 1516 can electrically communicate with a luminal surface of the outlet port 1506.

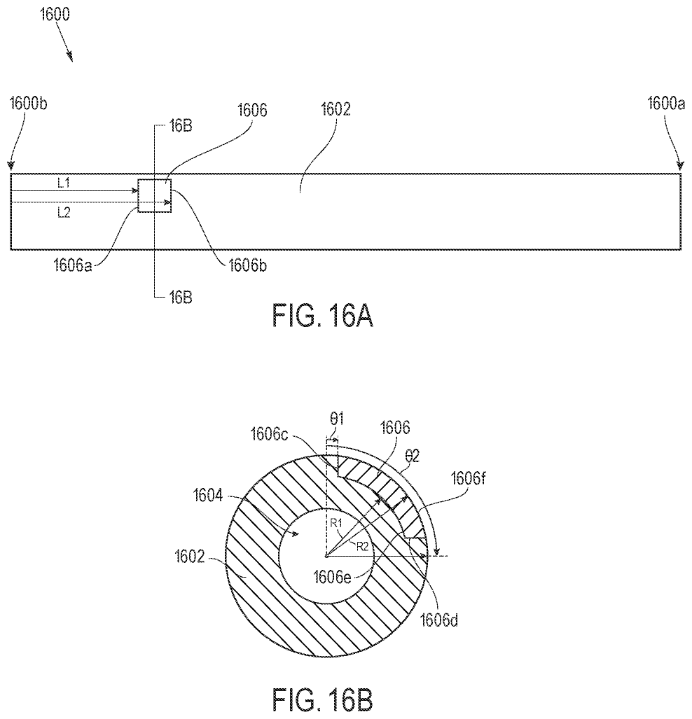

As described herein, a catheter in accordance with several embodiments of the present technology can carry one or more sensing elements configured to detect a physiological parameter. FIGS. 16A and 16B are side and cross-sectional views, respectively, of a catheter 1600 having a proximal end portion 1600a, a distal end portion 1600b, a sidewall 1602 defining a lumen 1604 of the catheter 1600 and a sensing element 1606 carried by the catheter 1600. The sensing element 1606 can be located at a specific position on the catheter 1600. As described herein, the position of the sensing element 1606 on the catheter 1600 can be based, at least in part, on a desired position of the sensing element 1606 with respect to an anatomic reference, a desired relative position between the sensing element 1606 and another electronic component on the catheter 1600 or another portion of the vascular access device, or another design consideration.

In some embodiments, for example as shown in FIGS. 16A and 16B, a sensing element 1606 can have a two-dimensional shape substantially corresponding to a rectangle and/or a three-dimensional shape substantially corresponding to a rectangular prism. In these and other embodiments, the sensing element 1606 can have a first edge 1606a spaced apart from the distal end 1600b of the catheter 1600 by a first longitudinal distance L1, a second edge 1606b spaced apart from the distal end 1600b by a second longitudinal distance L2, and a length defined between the first and second edges 1606a, 1606b. As shown in FIG. 16B, the sensing element 1606 can have a third edge 1606c at a first circumferential position θ1, a fourth edge 1606d at a second circumferential position θ2, and a width defined between the third and fourth edges 1606c, 1606d. The sensing element 1606 can have a fifth edge 1606e spaced apart from a central longitudinal axis L of the catheter 1600 by a first radial distance R1, a sixth edge 1606f spaced apart from the central longitudinal axis L by a second radial distance R2, and a thickness defined between the fifth and sixth edges 1606e, 1606f.

The sensing element 1606 can have any suitable two-dimensional shape (e.g., circular, triangular, hexagonal, etc.) or three-dimensional shape (e.g., cuboidal, spherical, cylindrical, conica, prismatic, toroidal, etc.). In these and other embodiments, a position of the sensing element 1606 on the catheter 1600 can be defined by points or edges forming a perimeter of the sensing element 1606, by a central point of a surface of the sensing element 1606, or by a central point within a volume of the sensing element 1606.

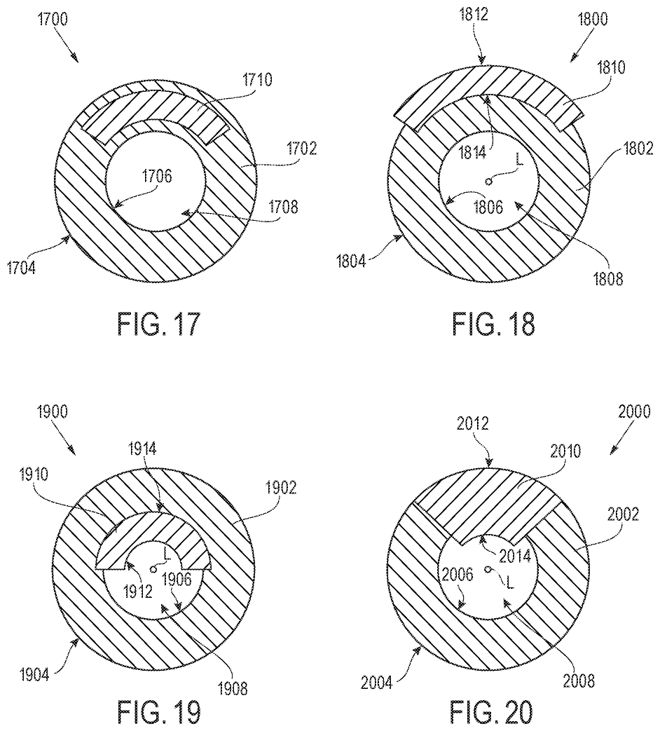

Although FIGS. 16A and 16B depict one sensing element 1606 located at one specific position, a catheter in accordance with the present technology can have any number of sensing elements located at any suitable positions. FIGS. 17-24 are axial cross-sectional views of example catheters carrying such sensing elements. These examples are intended to illustrate the variety of sensor configurations that are encompassed by the present technology and are not intended to be exhaustive. Any of the sensing elements and/or any other catheters shown in one of FIGS. 17-24 can be similar to any of the other sensing elements and/or catheters except as detailed below. Moreover, any of the sensing elements and any of the catheters can be combined with any other sensing elements and any other catheters disclosed herein and are not limited to the configurations shown in FIGS. 17-24. Each of the catheters 1700-2400 shown in FIGS. 17-24 has a sidewall 1702-2402 with an abluminal surface 1704-2404 and a luminal surface 1706-2406 defining a lumen 1708-2408 of the catheter 1700-2400. Each unique configuration depicted in FIGS. 17-24 is described in greater detail below.

As shown in FIG. 17, in some embodiments the catheter 1700 can carry a sensing element 1710 enclosed within the sidewall 1702 of the catheter 1700. The sensing element 1710 can be in intimate contact with the surrounding sidewall 1702 (see FIG. 17). Such configuration can be formed, for example, by molding the sidewall 1702 can be molded around the sensing element 1710. Although not depicted in FIG. 17, in some embodiments the sensing element 1710 is positioned within a channel defined by the sidewall 1702.

As shown in FIG. 18, the catheter 1800 can carry a sensing element 1810 positioned such that at least a portion of the sensing element 1810 is radially further from a central longitudinal axis L of the catheter 1800 than the abluminal surface 1804 of the catheter 1800. For example, the sensing element 1810 can be positioned on the abluminal surface 1804. In some embodiments the sensing element 1810 can be at least partially inset into the sidewall 1802 such that a first broad surface 1812 of the sensing element 1810 is radially further from the central longitudinal axis L than the abluminal surface 1804 but a second broad surface 1814 of the sensing element 1810 is radially closer to the central longitudinal axis L than the abluminal surface 1804.

In contrast to the catheter 1800 and sensing element 1810 of FIG. 18, the catheter 1900 shown in FIG. 19 carries a sensing element 1910 that is positioned such that at least a portion of the sensing element 1910 is radially closer to a central longitudinal axis L of the catheter 1900 than the luminal surface 1906 of the catheter 1900. The sensing element 1910 can be positioned on the luminal surface 1906. In some embodiments the sensing element 1910 is at least partially positioned within the sidewall 1902 of the catheter 1900 such that a first broad surface 1912 of the sensing element 1910 is radially closer to the central longitudinal axis L than the luminal surface 1906 but a second broad surface 1914 of the sensing element 1910 is radially further from the central longitudinal axis L than the luminal surface 1906.

The sensing elements 1710, 1810, and 1910 are each depicted as having a thickness (as defined with reference to FIGS. 6A and 6B) that is less than a thickness of the sidewall 1702, 1802, 1902 of the catheter 1700, 1800, 1900. However, as shown in FIG. 20, a sensing element 2010 can have a thickness greater than or equal to a thickness of the sidewall 2002 of the catheter 2000. Accordingly, the sensing element 2010 can have a first broad surface 2012 that is radially further from a central longitudinal axis L of the catheter 2000 than the abluminal surface 2004 of the catheter 2000 and/or a second broad surface 2014 that is radially closer to the central longitudinal axis L of the catheter 2000 than the luminal surface 2006 of the catheter 2000.

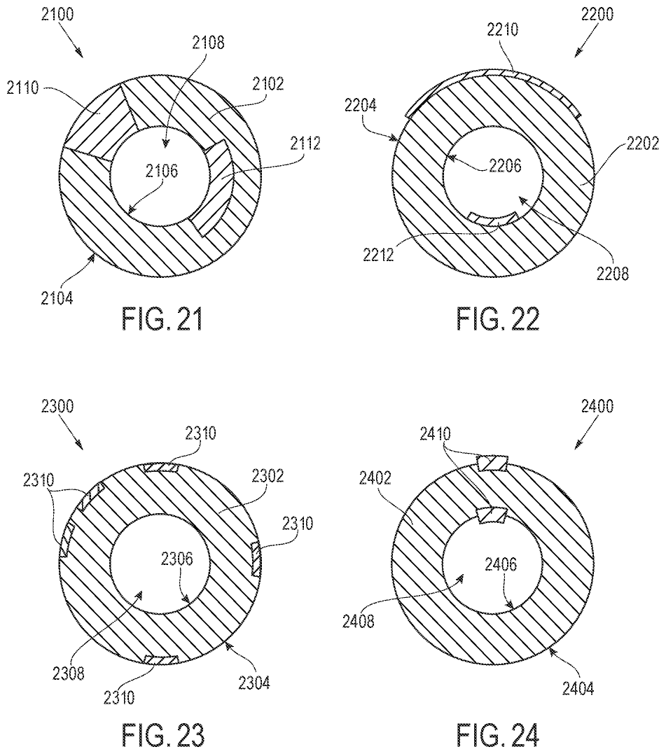

FIG. 21 depicts a catheter 2100 comprising a sidewall 2102, an abluminal surface 2104, a luminal surface 2106, and a lumen 2108. Although FIGS. 17-20 depict various catheters carrying a single sensing element, a catheter of the present technology can carry zero, one, or multiple sensing elements. For example, as shown in FIG. 21, the catheter 2100 can carry a first sensing element 2110 and a second sensing element 2112. In some embodiments, both the first and second sensing elements 2110, 2112 can be at least partially positioned within the sidewall 2102 of the catheter 2200. Although FIG. 21 depicts the first and second sensing elements 2110, 2112 having different widths and thicknesses (as defined with reference to FIGS. 6A and 6B), in some embodiments two sensing elements carried by a catheter can have substantially the same width (see FIG. 23) and/or thickness (see FIG. 24).

FIG. 22 depicts a catheter 2200 having a sidewall 2202 with an abluminal surface 2204 and a luminal surface 2206 defining a lumen 2208 of the catheter 2200. As shown in FIG. 22, one or more sensing elements can be positioned on one or more surfaces of the catheter 2200. For example, the catheter 2200 has a first sensing element 2210 positioned on the abluminal surface 2204 and a second sensing element 2212 positioned on the luminal surface 2206.

FIG. 23 depicts a catheter 2300 having a sidewall 2302 with an abluminal surface 2304 and a luminal surface 2306 defining a lumen 2308 of the catheter 2300. In some embodiments, multiple sensing elements carried by a catheter of the present technology can be circumferentially offset. For example, the catheter 2300 shown in FIG. 23 carries five sensing elements 2310, each of which is located at a unique position around the circumference of the catheter 2300. Moreover, as shown in FIG. 23, the sensing elements 2310 can be unevenly spaced around the circumference of the catheter 2300.

FIG. 24 depicts a catheter 2400 having a sidewall 2402 with an abluminal surface 2404 and a luminal surface 2406 defining a lumen 2408 of the catheter 2400. As shown in FIG. 24, in some embodiments the catheter 2400 can carry sensing elements 2410 that are circumferentially aligned.

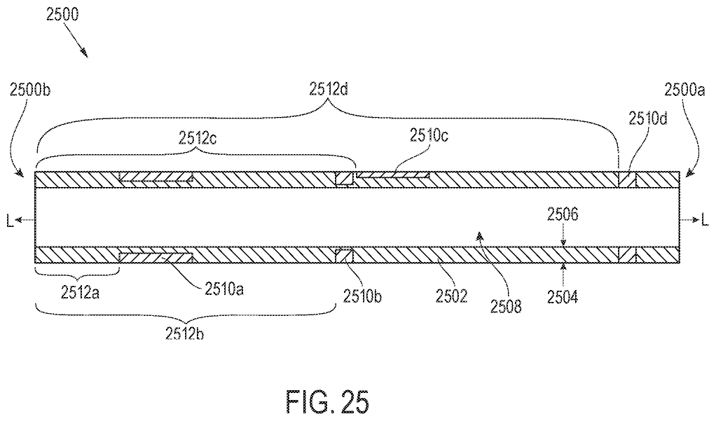

FIG. 25 is a cross-sectional side view of a catheter 2500 having a proximal end portion 2500a and a distal end portion 2500b. A sidewall 2502 of the catheter 2500 has an abluminal surface 2504, a luminal surface 2506 defining a lumen 2508 of the catheter. The catheter 2500 can carry one or more sensing elements. For example, FIG. 25 depicts a first sensing element 2510a, a second sensing element 2510b, a third sensing element 2510c, and a fourth sensing element 2510d (collectively “sensing elements 2510”). Each of the sensing elements 2510 can be circumferentially continuous (e.g., extending around the full circumference of the catheter) or circumferentially discontinuous (e.g., not extending around the full circumference of the catheter). For example, the first, second, and fourth sensing elements 2510a, 2510b, 2510d shown in FIG. 25 are circumferentially continuous while the third sensing element 2510c is circumferentially discontinuous. The sensing elements 2510 can be located at various longitudinal positions along the catheter 2500 defined by a distance 2512a-d between the distal end portion 2500b of the catheter 2500 and the respective sensing element 2510a-d. The sensing elements 2510 can be evenly spaced apart along the longitudinal axis L of the catheter 2500. Additionally or alternatively, at least some of the sensing elements 2510 can be unevenly spaced apart along the longitudinal axis L. For example, a longitudinal spacing between the first sensing element 2510a and the second sensing element 2510b shown in FIG. 25 is greater than a longitudinal spacing between the second sensing element 2510b and the third sensing element 2510c.

A spacing of the sensing elements 2510 with respect to the proximal end portion 2500a and/or distal end portion 2500b of the catheter 2500 can be selected based on the physiological parameter(s) to be detected by the sensing elements 2510. For example, it may be advantageous for a sensing element 2510 configured to detect a core temperature of a patient to be located as close to the distal terminus of the catheter 2500 as possible so that, when the device is implanted, the sensing element 2510 is located as centrally within the patient as possible. In some embodiments, spacing of the sensing elements 2510 relative to one another, a spacing of a sensing element 2510 relative to a reference on the device (e.g., a proximal or distal terminus of the catheter 2500, etc.), and/or a position of a sensing element 2510 relative to the patient's anatomy can be based on a desired performance of the sensing elements 2510. For example, it may be advantageous for sensing elements 2510 that are configured to detect an electrical signal of the patient's heart (e.g., EKG electrodes, etc.) to be spaced apart by no less than a predetermined threshold to ensure an electrical signal detected by the sensing elements 2510 is of sufficient quality.

As previously noted, in some embodiments it may be advantageous for sensing elements carried by a catheter of a vascular access device to be located at specific positions based on the patient's anatomy, a desired performance of the sensors, or another design consideration. For example, it may be advantageous for a sensing element configured to detect a central venous pressure of a patient to be located within at least one of the venae cavae and as close to the right atrium of the heart as possible. Accordingly, a vascular access device of the present technology can be configured such that, when the device is implanted, a central venous pressure sensing element is positioned within the superior vena cava or the inferior vena cava at a location as close to the right atrium as possible. In some embodiments, this can be ensured by spacing such a sensing element apart from the proximal terminus of the catheter and/or the housing defining the reservoir by a specific distance.

Additionally, it is often the case that the distal tip of a catheter of a vascular access device should be located at a specific position for delivery of medication and/or withdrawal of fluid samples via the device. For example, as noted with regards to FIGS. 4 and 5, in some cases it may be advantageous for the distal tip of the catheter to be positioned within a patient's superior vena cava, while in other cases it may be advantageous for the distal tip of the catheter to be positioned within a patient's inferior vena cava.

Despite the importance of precise and accurate positioning of the catheter and sensing elements within a patient, such positioning can be challenging to achieve. Patients have variable anatomy and the distance between the pocket in the upper chest wall where the hub is to be positioned and the desired location of the distal tip of the catheter or the desired location of the sensing element may differ across patients. Accordingly, even if two hubs are implanted at the exact same location in two different patients, the distal tip of the catheter and the sensing elements may be located at different positions within each of the patient's vasculature.

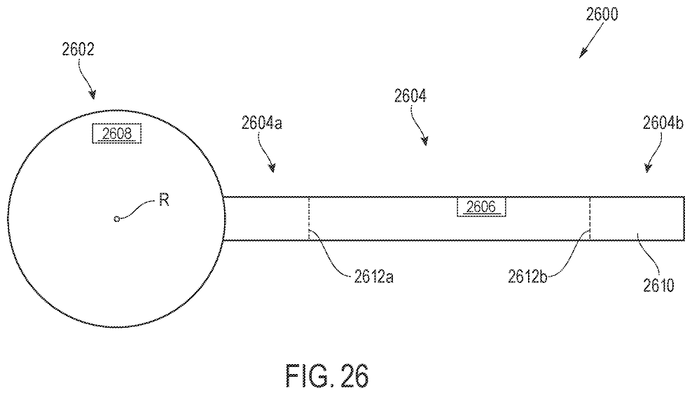

To address the above-noted needs and challenges, a vascular access device of the present technology can comprise a catheter whose length can be modified such that, when the device is implanted in a patient, the sensing element(s) are positioned at desired locations relative to the patient's anatomy. FIG. 26 schematically depicts a device 2600 comprising a hub 2602 and a catheter 2604. The catheter 2604 has a proximal end portion 2604a at the hub 2602 and a distal end portion 2604b configured to be positioned within the patient's vasculature and/or heart. The catheter 2604 can carry a sensing element 2606, which can be configured to be positioned at a specific location with respect to an anatomical reference and/or a reference on the hub 2602. In some embodiments, the catheter 2604 is configured such that a distance between the sensing element 2606 and a reference point R on the hub 2602 can be modified. For example, because the sensing element 2606 depicted in FIG. 26 is configured to wirelessly communicate with an electronic component 2608 carried by the hub 2602 and/or an extracorporeal electronic device, only one distinct portion of the catheter 2604 shown in FIG. 26 includes electronic components. Accordingly, the portions of the catheter 2604 that do not include any electronic components (e.g., the proximal end portion 2604a, the distal end portion 2604b, etc.) can be cut, torn, severed, melted, or otherwise modified to change a length of the portions without electronics. By modifying a length of the catheter 2604, a position of the sensing element 2606 relative to the reference point R on the hub 2602 can be modified.

In some embodiments, a sidewall 2610 of the catheter 2604 can include one or more markers 2612 configured to indicate information regarding the location of the sensing element 2606 and/or a desired length of the catheter 2604. For example, FIG. 26 illustrates a first marker 2612a proximal of the sensing element 2606 and a second marker 2612b distal of the sensing element 2606. The markers 2612 can be configured to indicate the location of the sensing element 2606 and thereby indicate portions of the catheter 2604 that can be modified and/or portions of the catheter 2604 that should not be modified. For example, the markers 2612 shown in FIG. 26 can indicate that a user should not cut the catheter 2604 between the markers 2612 to prevent damage to the sensing element 2606. Additionally or alternatively, the markers 2612 can be configured to indicate information for modifying a length of the catheter 2604 based on certain patient demographics (e.g., age, gender, height, ethnicity, etc.). In some embodiments, the markers 2612 can indicate locations to cut the catheter 2604 to reduce the length of the catheter 2604 based on the age of the patient. For example, the markers 2612 can indicate locations to cut the catheter 2604 when the catheter 2604 is to be used in a child under the age of 12. The locations of the markers 2612 can be based on average anthropomorphic measurements such that, once the shortened catheter 2604 is implanted in the patient, the distal tip of the catheter 2604 is positioned within the superior vena cava and near the right atrium.

As shown in FIG. 26, one or more of the markers 2612 can comprise visible indicia, which can be machine-readable and/or human-readable. In some embodiments, one or more of the markers 2612 can include at least one of numbers, letters, symbols, colors, shapes, or patterns. For example, as shown in FIG. 26, each of the first and second markers 2612a, 2612b includes a dashed line indicating where to cut the catheter 2604 and/or where not to cut the catheter 2604. The markers 2612 can indicate certain portions of the catheter 2604 (e.g., an intermediate portion carrying a sensing element or electronic component, a distal end portion configured to be trimmed, etc.). In some embodiments, one or more of the markers 2612 comprises a recess, an opening, or a protrusion in or on a sidewall of the catheter 2604. Additionally or alternatively, one or more of the markers 2612 can be formed by at least one of an ink, a film, a coating, a material, or a surface treatment of the catheter 2604. In one example, the catheter 2604 can be cut at the proximal and distal end portions 2604a, 2604b without damaging the sensing element 2606. In these and other embodiments, the proximal end portion 2604a and the distal end portion 2604b of the catheter 2604 can have a first visual appearance (e.g., a first color, etc.) and an intermediate portion 2604c of the catheter 2604 can have a second visual appearance (e.g., a second color, etc.) that is different from the first visual appearance such that a user can readily identify the portions of the catheter 2604 that can be cut without damaging the sensing element 2606.

In some embodiments, the device 2600 can be provided to a user in a pre-assembly configuration in which the catheter 2604 is not fixedly secured to the hub 2602. In such embodiments, the proximal end portion 2604a and/or the distal end portion 2604b of the catheter 2604 can be trimmed, cut, or otherwise modified and the catheter 2604 can be secured to the hub 2602 after modifying either end of the catheter 2604. However, in some embodiments the device 2600 can be provided to a user in an assembled configuration in which the proximal end portion 2604a of the catheter 2604 is fixedly secured to the hub 2602. In such embodiments, the distal end portion 2604b of the catheter 2604 can be modified, but not the proximal end portion 2604a.

As previously noted, a catheter of the present technology can carry one or more electrical components, such as a sensing element, configured to communicate with other electrical components via a wired connection. For example, as described with reference to FIGS. 7A-7C, in some embodiments a catheter can include a conductive element extending from a sensing element carried by the catheter to the hub. Accordingly, trimming the catheter at locations in which the catheter includes a conductive element would sever the electrical connection. Thus, in some embodiments, only the distal end portion of the catheter can be trimmed, regardless of whether the device is provided to a user in a pre-assembly configuration or an assembled configuration.

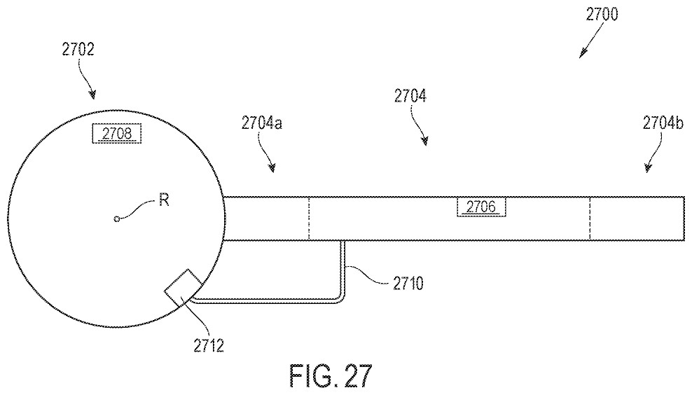

FIG. 27 schematically depicts a vascular access device 2700 comprising a hub 2702 and catheter 2704. The catheter 2704 has a proximal end portion 2704a at the hub 2702 and a distal end portion 2704b opposite the proximal end portion 2704a. The catheter 2704 can carry a sensing element 2706 which is configured to electrically communicate with an electronic component 2708 carried by the hub 2702. The electronic component 2708 can comprise a sensing element, a controller, a power source, or any other suitable electronic component as described herein. As shown in FIG. 27, the catheter 2704 can comprise a conductive element 2710 and the hub 2702 can comprise a connector 2712, which can be electrically coupled to the electronic component 2708 carried by the hub 2702. The connector 2712 can comprise a wire to wire connector, a wire to board connector, or another suitable connector.

The conductive element 2710 can have a distal end portion electrically coupled to the sensing element 2706 and a proximal end portion electrically coupled to the connector 2712. The distal portion of the conductive element 2710 can be coextruded with a sidewall of the catheter 2704, positioned within a lumen and/or channel of the catheter 2704, etc. Additionally or alternatively, the conductive element 2710 can have a proximal portion that is separate from the catheter 2704 (see FIG. 27). For example, the distal portion of the conductive element 2710 can be positioned within a channel defined by a sidewall of the catheter 2704 and the proximal portion of the conductive element 2710 can be positioned external of the catheter 2704. The conductive element 2710 can exit out of the channel of the catheter 2704 via an aperture in the sidewall. Accordingly, the catheter 2704 can be cut or otherwise modified at any location proximal of the point at which the conductive element 2710 exits the catheter 2704 without damaging the conductive element 2710.

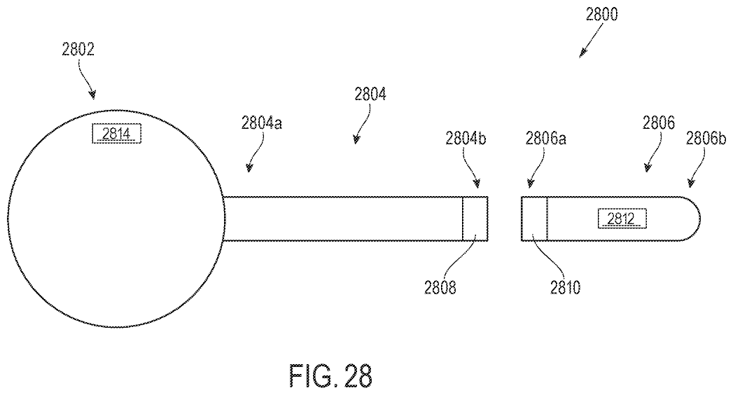

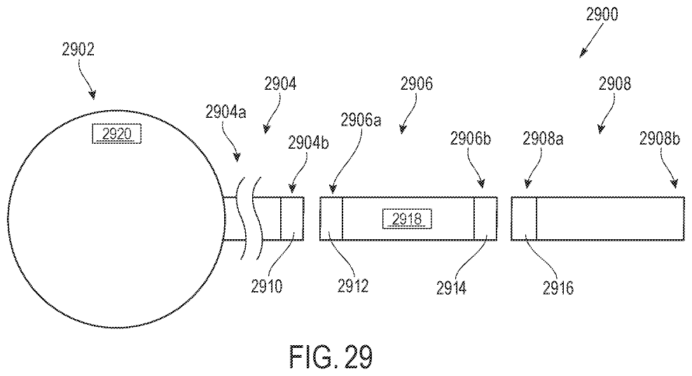

According to various embodiments, for example as shown in FIGS. 28 and 29, a vascular access device of the present technology can comprise a distinct electronics module that is configured to be secured to the catheter and/or hub. As shown in FIG. 28, a device 2800 can comprise a hub 2802, which is configured to be secured to a catheter 2804 having a proximal end portion 2804a and a distal end portion 2804b, which is configured to be secured to a proximal end portion 2806a of an electronics module 2806. The electronics module 2806 can comprise an atraumatic distal end portion 2806b to prevent or limit damage to the patient's vasculature and/or heart as a result of contact with the module 2806. The distal end portion 2804b of the catheter 2804 can include a securing element 2808 and/or the proximal end portion 2806a of the module 2806 can include a securing element 2810. In some embodiments, the securing element 2808 carried by the distal end portion 2804b of the catheter 2804 comprises a male fastener and the securing element 2810 carried by the proximal end portion 2806a of the module 2806 comprises a female fastener, or vice versa. Each of the securing elements 2808, 2810 can comprise a screw, a pin, a latch, adhesive, or another mechanism or material for joining components together. The electronics module 2806 can be configured to be releasably or permanently secured to the catheter 2804.

The distinct nature of the electronics module 2806 and catheter 2804 can facilitate and/or enable modifications of the length of the catheter 2804. For example, in some embodiments the proximal end portion 2804a and/or the distal end portion 2804b of the catheter 2804 can be cut or otherwise modified prior to securing of the electronics module 2806 to the catheter 2804. In some embodiments, the electronics module 2806 can be secured to the distal end portion 2804b of the catheter 2804 prior to trimming of the catheter 2804, such that only the proximal end portion 2806a of the catheter 2804 can be trimmed once the module 2806 and catheter 2804 have been secured.

The electronics module 2806 can include one or more electronic components 2812 including, but not limited to, a sensing element, a controller, memory, a data communications unit, or a power source. If the electronics module 2806 includes multiple electronic components 2812, these components can be communicatively coupled to one another via a wired or wireless connection. Additionally or alternatively, one or more of the electronic components 2812 can be configured to wirelessly communicate to an external device. In some embodiments, for example as shown in FIG. 28, the hub 2802 carries an electronic component 2814 which the electronic components 2812 of the module 2806 can be configured to electrically communicate with via wireless or wired connection.

The electronics module 2806 can be substantially cylindrical in shape. In some embodiments, an outer diameter of the electronics module 2806 is substantially equivalent to an outer diameter of the catheter 2804. The electronics module 2806 can have a lumen extending therethrough and sidewall openings in fluidic communication with the lumen such that fluids can be delivered or withdrawn through the electronics module 2806. In some embodiments, the electronics module 2806 is not configured for fluid delivery or withdrawal and does not have a lumen and/or sidewall openings, in which case the catheter 2804 would contain a side port for fluid communication between the catheter lumen and the surrounding bodily fluid.

FIG. 29 depicts a device 2900 comprising a hub 2902, a first catheter 2904 having a proximal end portion 2904a and a distal end portion 2904b, and an electronics module 2906 having a proximal end portion 2906a and a distal end portion 2906b. The device 2900 and any portion thereof can be similar to the device 2800 and any corresponding portion thereof, except as described below. As shown in FIG. 29, the proximal end portion 2906a of the electronics module 2906 can be configured to be secured to the distal end portion 2904b of the first catheter 2904. However, rather than the electronics module 2906 forming the distal end of the device 2900, a distal end portion 2906b of the electronics module 2906 can be configured to be secured to a proximal end portion 2908a of a second catheter 2908, such that a distal end portion 2908b of the second catheter 2908 forms an atraumatic distal end of the device 2900. The distal end portion 2904b of the first catheter 2904 can include a securing element 2910 configured to engage the proximal end portion 2906a of the module 2906 either directly or indirectly by engaging a securing element 2912 carried by the proximal end portion 2906a. The distal end portion 2906b of the module 2906 can include a securing element 2914 configured to engage the proximal end portion 2908a of the second catheter 2908 either directly or indirectly by engaging a securing element 2916 carried by the proximal end portion 2908a. The electronics module 2906 can have a lumen extending therethrough to permit fluid passage through the electronics module 2906 and to fluidically couple the lumen of the first catheter 2904 to the lumen of the second catheter 2908. The electronics module 2906 can carry one or more electronic components 2918, which can be communicatively coupled (e.g., physically, wirelessly, etc.) to one or more electronic components 2920 carried by the hub 2902.

In some embodiments, a system of the present technology can be configured to determine a physiological parameter of a patient based on data obtained by a vascular access device characterizing a parameter of the device. For example, because a catheter of an implanted vascular access device moves in a characteristic pattern throughout a patient's cardiac cycle, data characterizing motion of the catheter can be used to determine a heart rate parameter of the patient, a cardiac contractility parameter of the patient, a cardiac output parameter of the patient, or another parameter of the patient that is indicative of the patient's cardiac function.

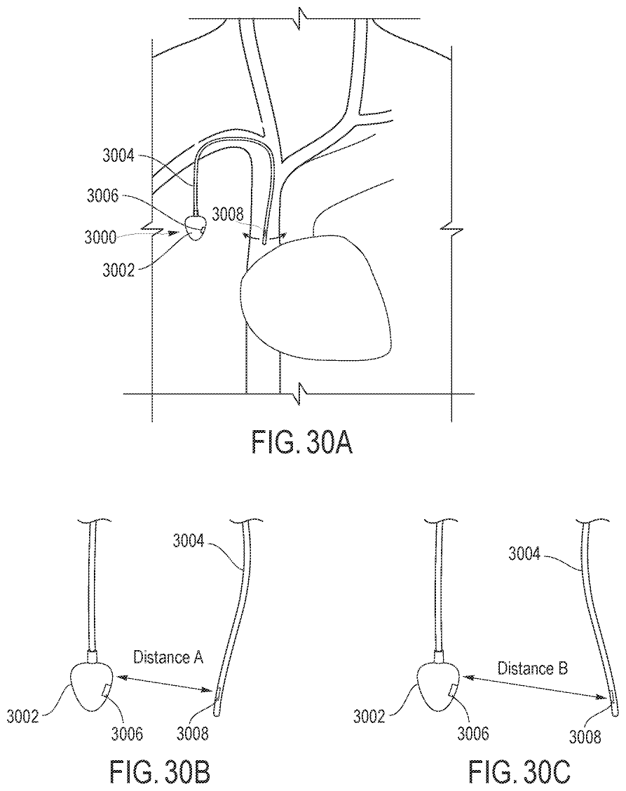

FIGS. 30A-30C schematically depict an example vascular access device 3000 (or “device 3000”) comprising a hub 3002 and an elongated member 3004. The elongated member 3004 can comprise a catheter, a wire, a rod, a tube, etc. As shown in FIG. 30A, the elongated member 3004 can be detachably or fixedly secured to the hub 3002. When the device 3000 is implanted, at least a portion the elongated member 3004 is positioned within at least one of the patient's vena cavae and/or the patient's right atrium. In some embodiments, the elongated member 3004 is fluidically coupled to the hub 3002.

The device 3000 can be configured to obtain data characterizing displacement and/or deformation of the elongated member 3004, which can be utilized to determine certain physiological parameters of a patient. The hub 3002 can carry one or more first sensing elements 3006 and the elongated member 3004 can carry one or more second sensing elements 3008. Although FIGS. 30A-30C depict the hub 3002 carrying a single first sensing element 3006 and the elongated member 3004 carrying a single second sensing element 3008, other numbers and configurations of first and second sensing elements 3006, 3008 are possible. As shown in FIGS. 30B and 30C, motion of the elongated member 3004, such as deflection of the distal end portion of the elongated member 3004, can cause the first and second sensing elements 3006, 3008 to be spaced apart by a varying distance. One or more of the first and second sensing elements 3006, 3008 can be configured to detect the distance between the first and second sensing elements 3006, 3008, and can thereby determine a position and/or movement (e.g., motion parameter) of the elongated member 3004.

To obtain data characterizing motion of the elongated member 3004, the first sensing element 3006 can comprise a proximity sensor configured detect a distance between the first sensing element 3006 and the second sensing element 3008. The first sensing element 3006 can comprise any suitable proximity sensing modality such as, but not limited to, photoelectric, inductive, capacitive, Hall effect, ultrasonic, etc. In some embodiments, the second sensing element 3008 can comprise an emitter configured to emit sensing energy and the first sensing element 3006 can comprise a detector configured to detect the sensing energy emitted by the emitter. In these and other embodiments, the sensing energy detected by the first sensing element 3006 can be used to determine a distance between the first and second sensing elements 3006, 3008. Additionally or alternatively, the second sensing element 3008 can comprise a passive target and the first sensing element 3006 can comprise a detector configured to detect the second sensing element 3008. For example, the first sensing element 3006 can comprise an inductive proximity sensor having an oscillation circuit and a coil configured to emit a magnetic field. The second sensing element 3008 can comprise a ferrous and/or conductive material that modifies the magnetic field in a distance-dependent manner. As the second sensing element 3008 moves closer to the first sensing element 3006, eddy currents are progressively generated in the second sensing element 3008, which in turn progressively create an opposing magnetic field that reduces the inductance of the inductive proximity sensor.

Still, in some embodiments, the second sensing element 3008 can comprise a proximity sensor configured to detect a distance between the second sensing element 3008 and the first sensing element 3006. As detailed above with respect to the first sensing element 3006, the second sensing element 3008 can comprise any suitable proximity sensing modality and the first sensing element 3006 can be an emitter or a passive target.

According to various embodiments, both the first sensing element 3006 and the second sensing element 3008 can comprise proximity sensors. The first and second sensing elements 3006, 3008 can be configured to detect one another and/or the first sensing element 3006 and/or the second sensing element 3008 can be configured to detect a target. For example, both the first and second sensing elements 3006, 3008 can comprise inductive proximity sensors configured to detect a passive target that is positioned on the vascular access device 3000, separate from the vascular access device 3000 but implanted within the patient, or external to the patient. The first and second sensing elements 3006, 3008 can comprise the same sensing modality or different sensing modalities. Additionally or alternatively, the first and second sensing elements 3006, 3008 can be configured to detect the same target or different targets.

In some embodiments, one or both of the first and second sensing element 3006, 3008 can comprise an emitter and/or a target configured to be detected by an extracorporeally-positioned detector. For example, the first and second sensing elements 3006, 3008 can comprise conductive material configured to be detected by an inductive proximity sensor positioned on the patient (e.g., on the patient's chest, on the patient's back, etc.) and the first and second sensing elements 3006, 3008 can comprise conductive material configured to be detected by the proximity sensor (e.g., by modifying the magnetic field generated by the proximity sensor as detailed herein). In these and other embodiments, displacement of the first and second sensing elements 3006, 3008, and thereby motion of the catheter, can be detected by the proximity sensor. The external proximity sensor can be secured to the patient (e.g., via medical tape, a bandage, etc.) to continuously obtain data characterizing motion of the catheter and/or the proximity sensor can be intermittently positioned

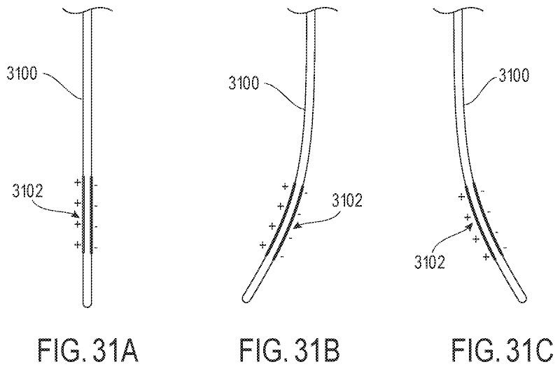

According to various aspects of the present technology, motion of a catheter can be characterized without determining a distance between the catheter and a hub of the vascular access device. For example, FIGS. 31A-31C schematically depict a distal end portion of an elongated member 3100 (e.g., a catheter, a wire, a rod, a tube, etc.) carrying one or more sensing elements 3102 configured to obtain data characterizing deformation of the distal end portion. In some embodiments, the sensing element 3102 can comprise a strain gauge whose resistance is configured to change in response to deformation of the distal end portion. In these and other embodiments, contraction of a patient's heart can cause motion of the distal end portion of the elongated member 3100, which can in turn cause a change in resistance of the sensing element 3102. The sensing element 3102 can comprise a foil gauge, a piezoresistor, a microscale strain gauge, a capacitive strain gauge, a vibrating wire strain gauge, a quartz crystal strain gauge, or another suitable gauge. The sensing element 3102 can comprise a linear strain gauge, a membrane rosette strain gauge, a double linear strain gauge, a full bridge strain gauge, a shear strain gauge, a half bridge strain gauge, a column strain gauge, a 45 degree rosette strain gauge, a 90 degree rosette strain gauge, or another suitable strain gauge.

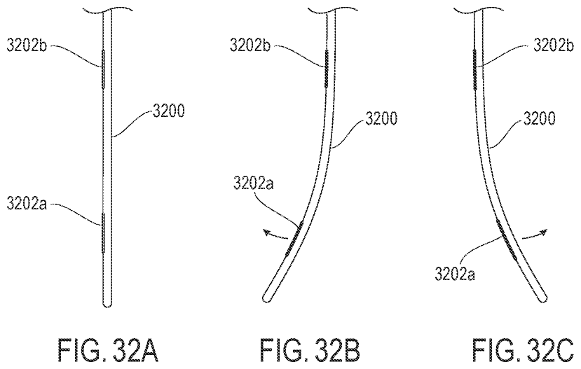

In some embodiments, the catheter can comprise multiple sensing elements spaced around a circumference of the catheter and/or along a length of the catheter. For example, FIGS. 32A-32C show an elongated member 3200 (e.g., a catheter, a wire, a rod, a tube, etc.) comprising first and second sensing elements 3202a, 3202b spaced apart along its distal portion. In some embodiments, the sensing elements 3202 can comprise linear strain gauges configured to obtain data characterizing deformation of the elongated member 3200 in a single direction. If two sensing elements 3202 are carried by the elongated member 3200, when the distal end portion of the elongated member 3200 deflects away from a longitudinal axis of the elongated member 3200 in a first direction, a first sensing element 3202a can be placed in compression while a second sensing element 3202b can be placed in tension. When the distal end portion of the elongated member 3200 deflects away from the longitudinal axis of the elongated member 3200 in a second, opposite direction, the first sensing element 3202a can be placed in compression while a second sensing element 3202b can be placed in tension. In this manner, bending of the elongated member 3200 and deflection of the distal portion can be characterized.

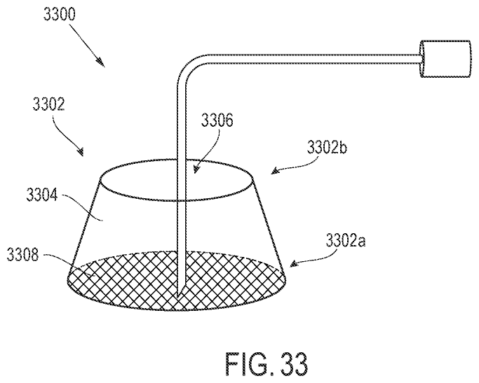

FIG. 33 schematically depicts a vascular access device 3300 (or “device 3300”) including a hub 3302. The device 3300 can include a catheter fluidically coupled to the hub 3302 (not shown). The hub 3302 can comprise a housing 3304 defining a reservoir 3306, and can have the same or similar features as any of the hubs disclosed herein, except as detailed below. As shown in FIG. 33, the hub 3302 can include a sensing element 3308. Although one sensing element 3308 positioned near a base portion 3302a of the hub 3302 is depicted in FIG. 33, any number or combination of sensing elements 3308 are possible. For example, the hub 3302 can carry one, two, three, four, five, six, seven, eight, nine, ten, or more sensing elements 3308. One or more sensing elements 3308 can be positioned at or adjacent the base portion 3302a of the hub 3302, at or adjacent to a top portion 3302b of the hub 3302, and/or between the base portion 3302a and the top portion 3302b. The sensing element 3308 can be partially or entirely positioned within the housing 3304, partially or entirely positioned within the reservoir 3306, partially or entirely on the housing 3304, etc.

The sensing element 3308 can be configured to obtain data characterizing a physiological parameter of the patient, a performance parameter of the device, a parameter of a treatment, or another parameter as described herein. Examples embodiments of the sensing element 3308 are detailed below. These examples are not intended to be exhaustive, but rather illustrate the various modalities, configurations, and functions of sensing elements of the present technology.

In some embodiments, the sensing element 3308 carried by the hub 3302 can be configured to obtain data characterizing a parameter of a needle configured to transport fluid in or out of the reservoir 3306 such as, but not limited to, data characterizing a presence of a needle within the reservoir 3306, data characterizing a position of a needle relative to a specific reference point on the hub 3302 (e.g., a distance between a distal tip of a needle and a septum covering the reservoir 3306 or a base of the reservoir 3306, etc.), data characterizing a material of the needle, etc. For example, the sensing element 3308 can comprise a pressure transducer positioned within the reservoir 3306 at the base portion 3302a of the hub 3302. The sensing element 3308 can be configured to obtain data characterizing a pressure within the reservoir 3306 such that, when a needle is inserted into the reservoir 3306 and contacts the sensing element 3308, a pressure detected by the sensing element 3308 increases. Additionally or alternatively, the sensing element 3308 can comprise a conductive element such that, when a conductive (e.g., metal) needle is inserted into the reservoir 3306 and contacts the sensing element 3308, an electrical circuit is opened or closed. In some embodiments, the sensing element 3308 can comprise an inductive proximity sensor configured to emit a magnetic field that is modified by a conductive object, such as a metallic needle, in a distance-dependent manner such that the sensing element 3308 is configured to measure a distance to the needle. A system of the present technology can use any of the previously-described data to determine whether the needle is properly inserted into the reservoir 3306 and whether it is safe to proceed with delivery or aspiration of fluid through the needle.

The sensing element 3308 can be configured to detect the presence of an object, such as a needle, within the reservoir 3306 and/or a position of an object within the reservoir 3306. It may be advantageous for the sensing element 3308 to determine a presence and/or position of an object, such as a needle, relative to the reservoir 3306 of the hub 3302 without contacting the object, for example so that the sensing element 3308 can be contained within the housing 3304 of the hub 3302 and is not exposed to fluids in the reservoir 3306. Accordingly, the sensing element 3308 can comprise a non-contact proximity sensor such as a photoelectric proximity sensor, a magnetic proximity sensor, an inductive proximity sensor, etc. Still, in some embodiments, the sensing element 3308 comprises an electromechanical sensor such as a linear variable differential transducer, a pressure transducer, a switch, etc. A sensing element 3308 comprising a pressure transducer can comprise a pressure-sensitive film, a force sensitive resistor, a strain gauge, a solid state transducer, a wet pressure sensor, or any other suitable pressure transducer.

In some embodiments, the sensing element 3308 can be configured to obtain data characterizing an indicator carried by the needle that is configured to communicate information regarding a patient or a treatment. For example, the sensing element 3308 can be configured to read machine-readable indicia such as a label, barcode, Quick Read (QR) code, iQR code, micro QR code, FrameQR code, or other machine-readable indicia carried by the needle. Such indicia can contain information regarding an identity of the patient, a type of the needle, injection system including needle and catheter system appropriate for power injection, a type of fluid to be transported through the needle, a location of treatment, a date of treatment, a time of treatment, or other patient information, device information, or treatment information. The sensing element 3308 can be configured to transmit data characterizing the information to a computing device for storage, interpretation by the computing device, etc.

The sensing element 3308 carried by the hub 3302 can be configured to obtain data characterizing one or more parameters such as, but not limited to, pressure within the reservoir 3306, flow rate within the reservoir 3306, temperature within the reservoir 3306, temperature of the hub 3302, temperature of the patient's tissues surrounding the hub 3302, presence and/or position of an object in the reservoir 3306, constituents and/or properties of fluid within the reservoir 3306, others, or combinations thereof.

In some embodiments, it may be advantageous for a hub of a vascular access device to comprise a first portion defining the reservoir and a second portion carrying electronic components, and for the first and second portions to be physically separable. For example, if a patient completes intravascular therapy but a clinician wishes to continue remote monitoring of the patient's health, it may be desirable to remove the catheter and reservoir from the patient while leaving the portion of the device containing the electronics implanted in the patient. A clinician and/or patient may want to remove the catheter and reservoir due to risks of complications occurring from long-term indwelling and/or the hassle of maintenance (e.g., monthly flushes and heparin locks, etc.). Moreover, the ability to remove the catheter and/or reservoir from the patient enhances the safety of the device, as either component can be removed if infection, thrombosis, or another illness were to occur while the device was implanted. Additionally or alternatively, it may be desirable to remove the electronics from the patient while leaving the catheter and reservoir implanted.

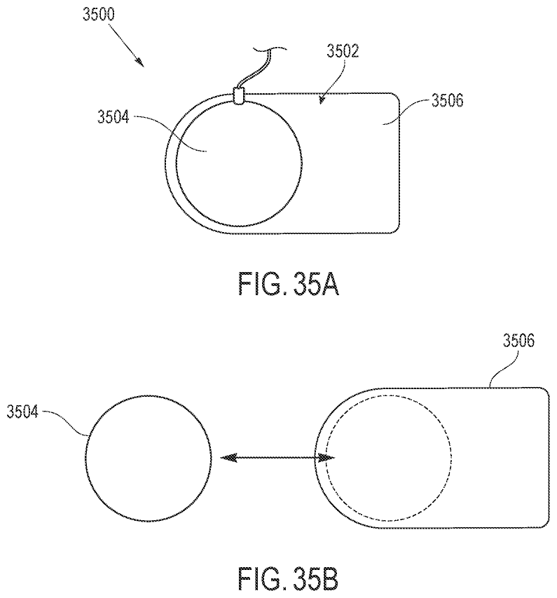

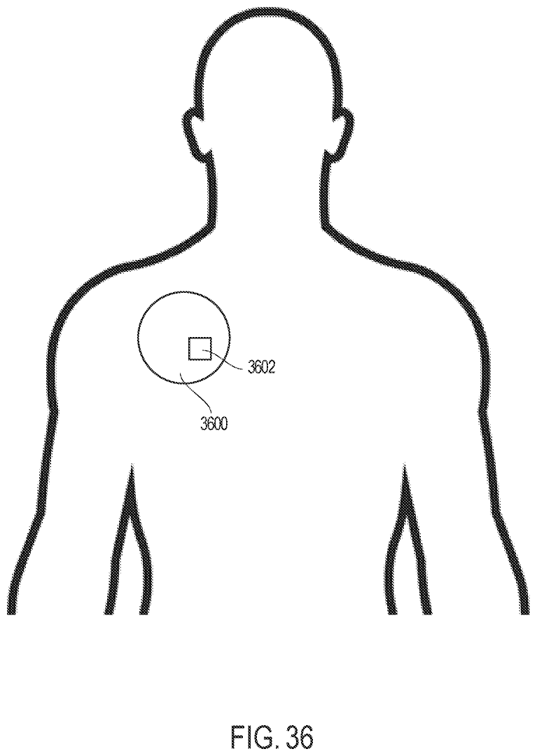

FIGS. 34A-34B and 35A-35B schematically depict examples of vascular access devices 3400, 3500 with such a configuration. As shown in FIG. 34A, the device 3400 can comprise a hub 3402 including a reservoir portion 3404 and an electronics portion 3406 secured to the reservoir portion 3404. In some embodiments, the reservoir portion 3404 and the electronics portion 3406 are both encased within a polymeric (e.g., silicone, polyurethane, thermoplastic elastomer, etc.) housing including a weakened portion 3408. The weakened portion 3408 can comprise perforations in the housing, a portion of the housing having a reduced thickness, or otherwise comprise a structurally compromised region such that the housing can be easily flexed, broken, torn, and/or cut at the weakened portion 3408 to produce a distinct reservoir portion 3404 and a distinct electronic portion 3406 (as shown in FIG. 34B). The device 3500 shown in FIGS. 35A-35B comprises a reservoir portion 3504 detachably secured to an electronics portion 3506. In some embodiments, an overmold contains the electronics portion, and the reservoir portion can be secured to and/or removed from the overmold. The reservoir portion 3504 can be secured to the electronics portion 3506 via one or more joining elements (e.g., snaps, screws, etc.), adhesive, or another suitable joining means.

According to several embodiments of the present technology, a vascular access device (or component thereof) can be configured to measure radiation exposure. For example, any of the vascular access devices disclosed herein can include a sensing element configured to detect and/or measure radiation. The sensing element can provide a continuous measurement of cumulative dose and current dose rate, and can warn the patient or healthcare team with an audible alarm when a specified dose rate or a cumulative dose is exceeded. In some embodiments, the sensing element only measures radiation intermittently, or on demand. The sensing element can be carried on and/or in the hub, or may be a separate component communicatively coupled to the hub and/or another component of the system. In some embodiments, for example, the vascular access device includes an integrated dosimeter. The integrated dosimeter can be an ion chamber dosimeter, or may be a thermoluminescent diode (TLD) dosimeter. The TLD can be configured to measure ionizing radiation exposure by measuring the intensity of light emitted from a Dy or B doped crystal in the detector when heated. The intensity of light emitted is dependent upon the radiation exposure.

Many physiologic events create characteristic sounds in the patient's body that can provide insight into the patient's health. Listening to the internal sounds of the body (or “auscultation”) is often a fundamental part of clinical examination and diagnostic procedures. Typically, auscultation is performed by a highly trained medical professional using a stethoscope. To examine a patient's lungs and respiratory function, for example, a medical professional may listen to sounds transmitted from the patient's body via the stethoscope, differentiate multiple body sounds from one another (e.g., tracheal sounds from bronchial sounds from bronchovesicular sounds from vesicular sounds, breath sounds from cardiac sounds, etc.), mentally evaluate various parameters of a sound of interest (e.g., frequency, intensity, duration, number, etc.), and mentally compare the sound of interest to known corresponding sounds (e.g., normal breath sounds, crackling sounds characteristic of chronic obstructive pulmonary disorder, wheezing sounds characteristic of pneumonia, absence of sound characteristic of pneumothorax, etc.). The ability to discern and interpret the body's many diverse sounds in a clinically meaningful way takes years of practice and is difficult to master. Not surprisingly, deficiencies in auscultation skills have been widely reported in the medical literature.

Despite the challenges associated with training, auscultation remains an important tool for obtaining information regarding physiological events, pathological changes, specific medical conditions, and overall health of a patient. Accordingly, various embodiments of the present technology are directed towards devices and systems configured to obtain audio data from the patient's body and process the audio data to obtain information regarding a patient's health. Additionally or alternatively, a medical device and/or system of the present technology can be configured to obtain audio data to obtain information regarding a performance or operation of the device and/or a treatment of the patient with the device.

FIG. 36 schematically illustrates an implantable medical device 3600 comprising a sensing element 3602 configured to obtain audio data from the patient's body. The sensing element 3602 can be, for example, a microphone or any other transducer configured to convert sound waves into an electrical signal. The sensing element 3602 can be carried on, at, and/or within a housing of the device 3600. The device 3600 can include an electronics component, such as any of the electronics components described herein. For example, the device 3600 can include a controller configured to be communicatively coupled to the sensing element 3602. In some embodiments, the device 3600 does not include a controller and instead the sensing element 3602 is configured to wirelessly send data to an external controller. According to various embodiments, the sensing element 3602 can be configured to communicate with both a local controller and an external controller. As discussed herein, processing of the data acquired by the sensing element 3602 of the device 3600 can take place at a local controller, an external controller, or both (all variations are intended to be included in the term “the controller”). The controller can be configured to receive audio data from the sensing element 3602 and, based on processing of the audio data alone and/or in combination with physiological data from one or more other sensing elements, determine one or more physiological parameters indicative of the patient's health.

The medical device 3600 can be configured to be positioned intradermally, subcutaneously, within a body lumen (e.g., a blood vessel, a bile duct, an intestine, etc.), secured to a wall of an organ, or in any other suitable location. In some embodiments, the medical device 3600 can be configured to be positioned on an external surface of a patient's skin. The medical device 3600 can provide one or more therapeutic functions such as drug delivery, electrical stimulation, structural support, and others. In some embodiments, the medical device 3600 has one or more additional diagnostic functions, such as temperature monitoring, blood constituent monitoring, etc. As described in greater detail below, the medical device 3600 can be a vascular access device, such as any of the vascular access devices disclosed herein.

The audio data obtained by the sensing element 3602 can be used by the controller and/or a medical professional to identify a physiological event such as, but not limited to, contraction of a patient's heart, opening or closing of patient's heart valve, blood flow through a patient's cardiovascular system, inhalation of air into a patient's lung, exhalation of air from a patient's lungs, sneezing, wheezing, coughing, snoring, speaking, peristalsis, muscular contraction, or other physiological events. Additionally or alternatively, the audio data obtained by the sensing element 3602 can be used by the controller and/or a medical professional to determine and/or indicate one or more physiological parameters of the patient. For example, the data can be used to determine a heart rate parameter, a heart rhythm parameter, a respiratory rate parameter, a cardiac output parameter, a forced expiratory volume in one second parameter, a forced vital capacity parameter, a forced expiratory flow parameter, a tidal volume parameter, a forced inspiratory flow parameter, a peak expiratory flow parameter, or another parameter of a patient. Additionally or alternatively, the data and/or derived parameters can be used by a system of the present technology and/or a medical professional to determine the presence and/or extent of a medical condition that the patient is afflicted by. Such medical conditions can include, but are not limited to, pneumothorax, valve disease, arrhythmia, pericarditis, acute myocardial infarction, septal defects, endocarditis, congestive heart failure, chronic obstructive pulmonary disorder, pulmonary edema, pneumonia, interstitial lung disease (e.g., pulmonary fibrosis, late-stage COPD, etc.), atelectasis, vomiting, cough, orthopnea, pulmonary embolism, sleep apnea, pleural effusion, emphysema, epiglottitis, recurrent laryngeal nerve paralysis, glottic larynx cancer, stroke, brain tumors, and/or falls.

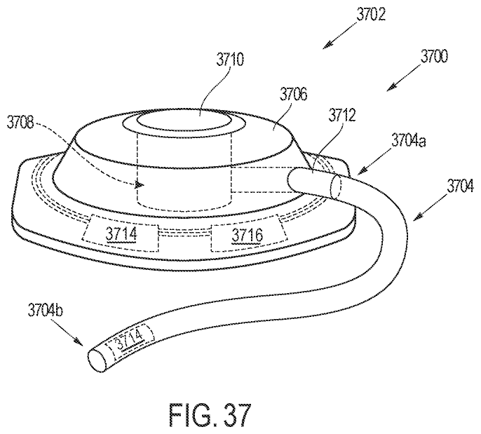

FIG. 37 schematically depicts a vascular access device 3700 (or “device 3700”) that is configured to obtain audio data. The device 3700 can comprise a hub 3702 and a catheter 3704 configured to be permanently or detachably coupled to the hub 3702. The catheter 3704 comprises a proximal end portion 3704a and a distal end portion 3704b. The hub 3702 can comprise a housing 3706, a fluid reservoir 3708 contained within the housing 3706, and a septum 3710 adjacent the reservoir 3708. In some embodiments, the catheter 3704 is secured to the hub 3702 via an outlet port 3712. The device 3700 can be configured to be implanted such that the hub 3702 is positioned in a subcutaneous pocket and the catheter 3704 is positioned within a blood vessel, such as the superior vena cava, or a heart of the patient.

As shown in FIG. 37, the device 3700 can comprise one or more sensing element(s) 3714 carried by the hub 3702 and/or catheter 3704 and configured to obtain audio data. In some embodiments, the sensing element 3714 comprises a microphone configured to configured to convert sound waves into an electrical signal. The device 3700 can comprise a controller 3716, carried by the hub 3702 and/or catheter 3704 and configured to be communicatively coupled to the sensing element(s) 3714. As described herein, the controller 3716 can be configured to receive audio data from the sensing element(s) 3714 and, based on processing of the audio data alone and/or in combination with data from one or more other sensing elements, determine one or more parameters indicative of the patient's health, operation of the device, performance of the device, a therapy delivered via the device, etc. In some embodiments, the device 3700 comprises one or more additional electronic components (e.g., a wireless communications module, a battery, etc.) which can be communicatively coupled to the sensing element(s) 3714 and/or the controller 3716.

In some embodiments, the sensing element(s) 3714 can be carried by the device 3700 at specific locations based, at least in part, on an intended purpose of the sensing element(s) 3714. For example, it may be advantageous for a sensing element 3714 configured to detect cardiac sounds to be carried by the distal end portion 3704b of the catheter 3704 so that, when the device 3700 is implanted, the sensing element 3714 is located near or within the heart. In another example, it may be advantageous to position a sensing element 3714 configured to detect respiratory sounds at the hub 3702 so that, when the device 3700 is implanted, the sensing element 3714 is located proximate the lungs and/or airways. Positioning a sensing element 3714 near the source of an acoustic wave to be detected can greatly improve the signal to noise ratio. Additionally or alternatively, a position and/or orientation of the sensing element(s) with respect to the device 3700 and/or the patient can be based, at least in part, on a directionality (e.g., a polar pattern) of the sensing element(s) 3714. For example, if the sensing element 3714 carried by the hub 3702 comprises a microphone having a cardioid polar pattern, the sensing element 3714 will only detect acoustic waves from one direction and should be positioned relative to the hub 3702 such that, when the device 3700 is implanted, the sensing element 3714 is oriented towards a source of an acoustic wave of interest (e.g., the heart to detect cardiac sounds, the lungs to detect respiratory sounds, etc.).

According to several embodiments, the controller 3716 can be configured to estimate a cardiac output parameter based at least in part on audio data acquired by the device 3700. The cardiac output parameter can be an estimate of cardiac output, a change in cardiac output, a characterization of output state (e.g., a high output, a low output states, etc.), or another suitable cardiac output parameter. In a person with a healthy heart and/or heart valves, blood flow out of the left ventricle and across the aortic valve is not audible through a stethoscope. When the blood flow increases, an audible murmur often arises because of the flow becoming turbulent. The murmur may get louder as the flow increases, which is sometimes referred to as a flow murmur or high output murmur. In some embodiments, the sensing element 3714 can be configured to obtain audio data characterizing blood flow over the aortic valve, and the controller 3716 can be configured to use the acquired audio data to characterize the flow (e.g., low flow versus high flow, etc.). This characterization can be used to determine if cardiac output is increasing or decreasing, and such a determination could be in many clinical scenarios. For example, if a patient has a fever and the controller 3716 indicates that the patient is experiencing an increase in blood flow based on audio data collected by the sensing element 3714, this can indicate that the patient is showing early signs of sepsis.

The controller 3716 can, in some embodiments, be configured to estimate a source of a murmur based at least in part on audio data acquired by the device 3700. A murmur can be indicative of valvular disease such as stenosis or insufficiency, which can occur at any of the four heart valves. To estimate the source of the murmur, the controller 3716 can use audio data to indicate whether the murmur occurs during systole or diastole and/or the portion of systole or diastole that the murmur occurs during (e.g., mid systole, holosystole, end systole, etc.), which can indicate the source of the murmur. In some embodiments, the controller 3716 can be configured to quantitatively characterize a parameter of the audio data (e.g., pitch, frequency, amplitude, intensity, etc.), which can indicate the source of the murmur. Additionally or alternatively, the controller 3716 can be configured to qualitatively characterize the sound. For example, the controller 3716 can characterize the sound as a “click,” which can be indicative of mitral valve prolapse. In some cases, a murmur can occur due to an atrial or ventricular septal defect. Accordingly, the controller 3716 can be configured to use the audio data to detect a murmur that is characteristic of an atrial septal defect and/or a ventricular septal defect, which can be used to indicate the source of the murmur.

In some embodiments, based on the audio data (alone or in combination with one or more other sensed parameters), the controller 3716 can be configured to detect a murmur from valvular dysfunction secondary to vegetation formation that is indicative of endocarditis. Additionally or alternatively, the controller 3716 can be configured to detect a characteristic sound of cardiac rub that is indicative of pericarditis.

The controller 3716 can be configured to characterize an abnormal heart sound and/or a change in a heart sound of a patient, which can indicate one or more cardiovascular conditions. According to various embodiments, the controller 3716 can be configured to detect an abnormal heartbeat of a patient. For example, the controller 3716 can be configured to characterize an abnormal cardiac sound and/or a change in a cardiac sound of a patient, which can indicate an abnormal heartbeat associated with bradycardia, tachycardia, premature atrial contractions, atrial fibrillation, atrial flutter, paroxysmal supraventricular tachycardia, accessory pathway tachycardia, AV nodal reentrant tachycardia, premature ventricular contractions, ventricular tachycardia, ventricular fibrillation, long QT syndrome, bradyarrhythmia, sinus node dysfunction, heart block, or another arrythmia.

In some embodiments, based on the audio data (alone or in combination with one or more other sensed parameters), the controller 3716 can be configured to characterize an abnormal cardiac sound and/or a change in a cardiac sound of a patient that is indicative of acute myocardial infarction.

Typical heart sounds of a healthy patient comprise a first heart sound (S1) corresponding to closing of the mitral and tricuspid valves and a second heart sound (S2) corresponding to closing of the aortic and pulmonic valves. Patients with heart failure often have a third heart sound (S3) and/or a fourth heart sound (S4), which are abnormal. Based on the audio data, the controller 3716 can be configured to determine the presence of abnormal heart sounds, which can indicate signs of heart failure.

In some cases, a patient's heart function and health can be estimated by evaluating non-cardiac sounds. For example, a patient with venous congestion will often develop pulmonary edema, which can cause crackles or “rales” in the lungs, wheezing, and/or changes in respiratory rate. Patients with congenital heart failure often experience orthopnea, or positional shortness of breath associated with an increase in pulmonary edema when the patient lies flat. Such increase in pulmonary edema can cause and/or exacerbate the abnormal breath sounds associated with pulmonary edema. As such, the controller 3716 can be configured to detect and/or characterize respiratory sounds and/or other body sounds that can be used to estimate a patient's cardiac function. The controller 3716 can be configured to use the audio data alone or in combination with another sensed parameter. For example, a position of a patient can be detected by an accelerometer and/or gyroscope which, when used by the controller 3716 in combination with audio data characterizing abnormal breath sounds characteristic of pulmonary edema, can be used to detect orthopnea.

Many pathologies and medical conditions can cause changes in a patient's breath sounds and/or abnormal breath sounds. The controller 3716, according to various embodiments of the present technology, can be configured to use the audio data (alone or in combination with one or more other sensed parameters) to detect such changes and/or abnormalities in a patient's breath sounds. For example, as noted above, the controller 3716 can be configured to detect crackles, rales, or wheezing in a patient's breath sounds, which can be indicative of pulmonary edema.

According to some embodiments, the controller 3716 can be configured to estimate pulmonary function testing parameters, such as forced expiratory volume during the first (FEV1), second (FEV2), and third (FEV3) seconds of a forced breath and/or forced vital capacity (FVC). FEV1 and FVC are often diminished in patients with obstructive lung disease like asthma and chronic obstructive pulmonary disease (e.g., emphysema, chronic bronchitis, etc.). When obstructive lung disease worsens, the expiratory phase of breathing is prolonged and the sound of wheezing increases. Based on the patient's baseline pulmonary function and obtained audio data, the controller 3716 can determine a time of the patient's expiratory phase and/or characterize the presence and extent of wheezing (e.g., by volume, pitch, etc.), which can be used to estimate changes in underlying lung function.

According to various embodiments, the controller 3716 can be configured to detect the presence and/or extent of a pneumothorax in a patient. A patient suffering from a pneumothorax will have an absence in breath sounds in the area of the pneumothorax. Thus, the controller 3716 can be configured to determine that there is an absence of an expected breath sound, which can indicate the presence of a pneumothorax. In some embodiments, the controller 3716 can be configured to characterize a degree of a loss of breath sounds, which can be used to estimate a size of the pneumothorax. In some embodiments, based on the audio data (alone or in combination with one or more other sensed parameters), the controller 3716 is configured to detect diminished or absent breath sounds in a region of atelectasis (obstructive or non-obstructive), diminished breath sounds in a region of a pleural effusion, and/or diminished breath sounds due to empyema.

Lobar pneumonia can lead to consolidation of one or more lobes of the lung, which will often cause a loss of breath sounds or “bronchiolar” breath sounds in which air passes through the large and medium airways, but not all the way to the alveoli. According to various embodiments of the present technology, the controller 3716 can be configured to detect a loss of breath sounds and/or bronchiolar breath sounds, which can be used to determine if a patient is suffering from lobar pneumonia and/or lobar consolidation. Lobar consolidation can also lead to egophony. Traditionally, egophony is detected when a medical professional performing auscultation asking a patient to say a long “e” sound and instead they produce an “a” sound. The controller 3716 can be configured to use audio data collected during such an exercise to detect such an abnormality in the patient's speech, which can be indicative of lobar pneumonia and/or consolidation.

The controller 3716 can, according to various embodiments, use the audio data either alone or in combination with one or more other sensed parameters to detect a pulmonary embolism. For example, a pulmonary embolism can cause strain on the right side of the heart, which can cause a right heart fourth heart sound (S4). In some cases, a pulmonary embolism can cause pulmonary edema which, as detailed above, can cause changes in respiratory rate and/or characteristic sounds such as crackles, rales, and/or wheezing.

In some embodiments, based on the audio data (alone or in combination with one or more other sensed parameters), the controller 3716 is configured to detect coarse or dry breath sounds, which can be used to detect interstitial lung disease (e.g., pulmonary fibrosis, late-stage chronic obstructive pulmonary disorder, etc.). As described above with reference to FIG. 36, a long term implantable device 3600 of the present technology can comprise a sensing element 3602 configured to obtain audio data, and the audio data can characterize a breath sound of a patient. In some embodiments, the device 3600 includes a controller configured to detect abnormal breath sounds and/or changes in breath sounds characteristic of interstitial lung disease. The device 3600 can be implanted in the posterior/lateral chest wall and/or directed at the posterior costophrenic sulci, which is where most of the changes occur with interstitial lung disease, as well as the accumulation of pleural effusions in some patients.

Anaphylaxis can cause stridor and/or wheezing upon inspiration as a result of upper airway and/or laryngeal edema due to anaphylaxis. Additionally or alternatively, a patient's cardiac output can increase due to anaphylaxis. Accordingly, a controller 3716 according to various aspects of the present technology can be configured to use the audio data to detect stridor, wheezing, and/or cardiac output, which can be used to detect anaphylaxis.

In some embodiments, based on the audio data (alone or in combination with one or more other sensed parameters), the controller 3716 is configured to detect the sound of snoring intermixed with absence of breath sounds while a patient is lying down (for example, as measured by an activity and/or position sensing element) which can be used to detect sleep apnea.

In some embodiments, based on the audio data (alone or in combination with one or more other sensed parameters), the controller 3716 is configured to detect slurred speech, which can be used to detect a stroke or a brain tumor.

The controller 3716 can be configured to detect cough based on the audio data (alone or in combination with one or more other sensed parameters). In some embodiments, the controller 3716 is configured to determine a parameter of a cough (e.g., a frequency, an intensity, dry, productive, etc.) from the audio data.

In some embodiments, based on the audio data (alone or in combination with one or more other sensed parameters), the controller 3716 is configured to detect abnormal vocal and/or gastrointestinal sounds, which can be used to detect vomiting. Additionally or alternatively, the controller 3716 can be configured to detect mastication based on the acquired audio data (e.g., based on vocal sounds, gastrointestinal sounds, etc.). In some embodiments, the controller 3716 can create and index the audio data to characterize mastication. For example, the controller 3716 can detect a time of mastication, which can be indicative of decreased appetite, hypogeusia, anorexia, etc.

The controller 3716 can, according to various embodiments, be configured to detect swallowing. For example, the controller 3716 can use the audio data, alone or in combination with one or more other sensed parameters, to detect vocal and/or gastrointestinal sounds of a patient to detect swallowing. Swallowing detection can be for monitoring a patient's consumption of food or liquids, a patient's nutritional status, a patient's compliance to a feeding or fluid regimen, etc. In some embodiments, the device 3700 can be configured to provide a notification (e.g., a tactile notification, a visible notification, an audible notification, etc.) to the patient based on the detected swallowing. For example, the device 3700 can provide a notification the patient to communicate that the patient should consume fluids. Such notification can be provided, for example, if the controller 3716 does not detect a swallow over a predetermined duration of time and/or if the controller 3716 detects tachycardia, which could be caused by dehydration. In some embodiments, the device 3700 can be configured to provide a notification to the patient to provide feedback regarding the patient's compliance to a treatment plan, a requested action, etc.

Various pathologies and medical conditions can cause changes in a patient's voice. For example, epiglottitis can cause stridor and/or “hot potato voice,” glottic larynx cancer can cause a hoarse voice, and recurrent laryngeal nerve paralysis, which can occur with mediastinal masses, can cause vocal changes. The controller 3716 can be configured to use the audio data to detect vocal changes or abnormalities, which can indicate one or more pathologies or medical conditions.

In some embodiments, the controller 3716 can be configured to use the audio data (alone or in combination with one or more other sensed parameters) to detect extrathoracic sounds. For example, the controller 3716 can be configured to detect abdominal and/or gastrointestinal sounds (or the lack thereof), such as hypoactive bowel sounds, hyperactive bowel sounds, borborygmi, flatulence, etc. Abnormal abdominal and/or gastrointestinal sounds can be indicative of obstruction, ileus, hernia, tumor, ulcers, bleeding, inflammatory bowel disease, Crohn's disease, infection, food allergies, peritonitis, etc. Additionally or alternatively, the controller 3716 can be configured to detect womb sounds of a pregnant woman during gestation, which can provide insight into the health of the woman and/or her child.

The controller 3716 can be configured to use the audio data (alone or in combination with one or more other sensed parameters) to detect a sound originating from outside of the patient's body. For example, the controller 3716 can be configured to detect sounds associated with scratching of the patient's skin, which can indicate a response to a medical treatment, an allergy, a dermatological disorder, a psychological disorder, etc. As a specific example, the controller 3716 can be configured to detect scratching of the skin secondary to pruritis, which can be a side effect of chemotherapy or hyperbilirubinemia.

In some embodiments, the controller 3716 can use the audio data (alone or in combination with one or more other sensed parameters) to detect an activity of a patient. For example, the controller 3716 can detect a sound associated with a patient falling, such as the sound of airflow, calls for help, or cries of pain. In some embodiments, the controller 3716 can use data from an accelerometer to facilitate the detection of an activity of the patient in combination with the audio data.

In some embodiments, the controller 3716 can be configured to use the audio data (alone or in combination with one or more other sensed parameters) to detect a parameter associated with a performance of the device 3700. For example, the controller 3716 can detect a parameter of the audio data (e.g., a presence of the data, an amplitude, an intensity, a frequency, a pitch, etc.) and indicate whether the device 3700 is performing as intended. In some embodiments, the controller 3716 can detect a change in the audio data over time, which can be indicative of a change in the impedance of the sensing element 3714.

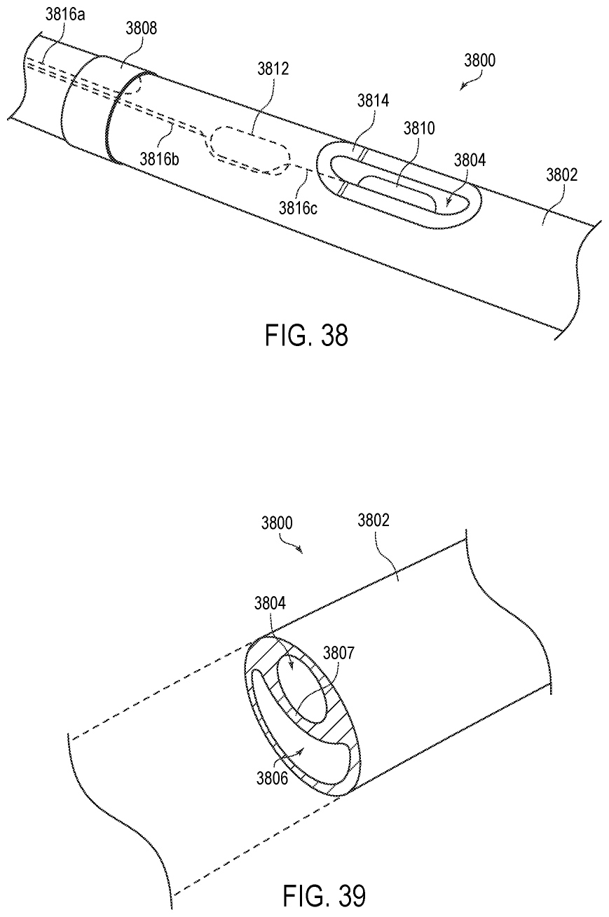

FIG. 38 is a perspective view of a portion of a catheter 3800 in accordance with the present technology. FIG. 39 is a partially transparent perspective view of a portion of the catheter 3800 with a transverse cross-section of the catheter 3800 highlighted. Among other things, FIGS. 38 and 39 illustrate several examples of different ways in which catheters in accordance with the present technology can incorporate electrodes and sensing elements. With reference to FIGS. 38 and 39 together, the catheter 3800 can include an elongate catheter body 3802 defining a first axial lumen 3804 and a second axial lumen 3806. Between the first and second axial lumens 3804, 3806, the catheter body 3802 can include a curved internal wall 3807. In at least some cases, the curved internal wall 3807 isolates the first and second axial lumens 3804, 3806 from one another all of a length of the catheter 3800. Alternatively, the curved internal wall 3807 can extend along only a portion of the length of the catheter 3800. For example, the first axial lumen 3804 or the second axial lumen 3806 can end distally and/or proximally before the distal and/or proximal ends of the catheter 3800. In these cases, the first axial lumen 3804 or the second axial lumen 3806 can be a cavity lumen rather than a through lumen.

The catheter 3800 can further include an annular electrode 3808 extending around a transverse perimeter of the catheter body 3802, a first sensing element 3810 within the first axial lumen 3804, and a second sensing element 3812 within the second axial lumen 3806. The catheter body 3802 can define a window 3814 opening into the first axial lumen 3804 and proximate to the first sensing element 3810. The catheter 3800 can further include electrical leads 3816a-3816c embedded in the internal wall 3807 or another wall portion of the catheter body 3802 and extending proximally from the annular electrode 3808, the second sensing element 3812, and the first sensing element 3810, respectively. In other embodiments, the first sensing element 3810, the second sensing element 3812, and the window 3814 can have different arrangements. For example, the window 3814 can open into the second axial lumen 3806 and/or be proximate to the second sensing element 3812. As another example, the second sensing element 3812 can be embedded in the internal wall 3807 or another wall portion of the catheter body 3802 rather than being within the second axial lumen 3806.

With reference again to FIGS. 38 and 39, the first axial lumen 3804 and the second axial lumen 3806 can have different purposes. For example, the first axial lumen 3804 can serve at least primarily for delivery of material into and/or extraction of material from a subject's bloodstream while the second axial lumen 3806 serves at least primarily for sensing and/or monitoring. In some cases, the first axial lumen 3804 and the first sensing element 3810 are configured to be exposed to the bloodstream via the window 3814. This can be useful, for example, when the first sensing element 3810 is a pressure transducer configured to sense a subject's blood pressure at a location laterally adjacent to the catheter 3800. In other cases, the window 3814 is an optically transparent portion of the catheter body 3802. This can be useful, for example, when the first sensing element 3810 is an optical element configured to receive and/or to convey an optical signal. As shown in FIG. 39, the first and second axial lumens 3804, 3806 can have different transverse cross-sectional shapes. In the illustrated embodiment, the first axial lumen 3804 has a round transverse cross-sectional shape and the second axial lumen 3806 has a rounded crescent transverse cross-sectional shape. In other embodiments, counterparts of the first axial lumen 3804 and the second axial lumen 3806 can have other suitable shapes.

### CONCLUSION

Although many of the embodiments are described above with respect to vascular access devices, the technology is applicable to other applications and/or other approaches, such as other types of implantable medical devices (e.g., pacemakers, implantable cardioverter/defibrillators (ICD), deep brain stimulators, insulin pumps, infusion ports, orthopedic devices, and monitoring devices such as pulmonary artery pressure monitors). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-37.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

For the purposes of this specification and appended claims, unless otherwise indicated, all numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

I/We claim:

1. A vascular access device comprising:
a hub comprising a housing defining a reservoir configured to receive a fluid therein;
a catheter comprising a proximal end portion configured to be secured to the hub, a distal end portion configured to be positioned within a cardiovascular system of a patient, and a lumen extending therethrough, the lumen being in fluid communication with the reservoir;
a sensing element carried by the catheter, the sensing element being configured to obtain data characterizing at least one of a physiological parameter of a patient or a performance parameter of the vascular access device; and
a data communications module communicatively coupled to the sensing element and configured to transmit the data obtained by the sensing element to an external computing device.

2. The vascular access device of claim 1, wherein the data communications module is carried by the housing of the hub.

3. The vascular access device of claim 1 or claim 2, wherein the sensing element is configured to communicate with the data communications module via a wireless connection.

4. The vascular access device of claim 1 or claim 2, further comprising a conductive element having a first portion electrically coupled to the sensing element and a second portion electrically coupled to the data communications module.

5. The vascular access device of claim 4, wherein the conductive element and catheter are coextruded.

6. The vascular access device of claim 4 or claim 5, wherein the conductive element is at least partially positioned within a sidewall of the catheter.

7. The vascular access device of any one of claims 4 to 6, wherein the catheter comprises a channel that is radially offset from the lumen, and wherein the conductive element is positioned within the channel.

8. The vascular access device of any one of claims 1 to 7, further comprising a battery in electrical communication with the sensing element.

9. The vascular access device of any one of claims 1 to 8, wherein the sensing element is at least partially positioned within a sidewall of the catheter.

10. The vascular access device of any one of claims 1 to 9, wherein the sensing element is at least partially exposed to the lumen of the catheter and/or an environment surrounding the catheter.

11. The vascular access device of any one of claims 1 to 10, wherein the lumen is a first lumen, the catheter having a second lumen radially offset from the first lumen.

12. The vascular access device of claim 11, wherein the sensing element is positioned within the second lumen.

13. The vascular access device of claim 11 or claim 12, wherein the first lumen is fluidically isolated from the second lumen.

14. The vascular access device of any one of claims 1 to 13, wherein the physiological parameter of the patient comprises at least one of a heart rate of the patient, a central venous pressure of the patient, a respiratory rate of the patient, a respiratory sound of the patient, a cardiac sound of the patient, a gastrointestinal sound of the patient, a speech of the patient, a core temperature of the patient, an electrical signal of a heart of the patient, an activity level of the patient, a blood oxygenation of the patient, or a blood glucose of the patient.

15. The vascular access device of any one of claims 1 to 14, wherein the performance parameter of the vascular access device comprises at least one of a flow rate within the lumen of the catheter, a pressure in the lumen of the catheter, a temperature of the catheter, an electrical impedance of the sensing element, or a charge level of a battery of the device.

16. A vascular access device comprising:
a hub comprising a housing defining a reservoir configured to receive a fluid therein;
a catheter comprising a proximal end portion secured to the hub, a distal end portion opposite the proximal end portion along a length of the catheter, an intermediate portion therebetween, and a sidewall defining a lumen fluidically coupled to the reservoir; and
a sensing element carried by the intermediate portion of the catheter, the sensing element being configured to obtain data characterizing at least one of a physiological parameter of a patient or a performance parameter of the vascular access device,
wherein the distal end portion of the catheter is separable from the intermediate portion of the catheter such that the length of the catheter is adjustable.

17. The vascular access device of claim 16, wherein the distal end portion of the catheter is configured to be cut to be separated from the intermediate portion of the catheter.

18. The vascular access device of claim 16 or claim 17, wherein the catheter comprises one or more markers configured to indicate a location of at least one of the intermediate portion of the catheter or the distal end portion of the catheter.

19. The vascular access device of claim 18, wherein the one or more markers comprise at least one of a film, a coating, a surface treatment, a recess, an opening, or a protrusion.

20. The vascular access device of claim 18 or claim 19, wherein the one or more markers comprise at least one of a number, a letter, a color, a symbol, a pattern, or a shape.
